(19) United States
(12) Patent Application Publication
(10) Pub. No.: US 2023/0109504 A1
(43) Pub. Date: Apr. 6, 2023

(54) GENE THERAPY TREATMENT

(71) Applicant: University of Sheffield, Sheffield (GB)

(72) Inventors: Mimoun Azzouz, Sheffield (GB); Joseph Scarrott, Sheffield (GB); Evangelia Karyka, Sheffield (GB)

(73) Assignee: University of Sheffield, Sheffield (GB)

(21) Appl. No.: 17/910,125

(22) PCT Filed: Apr. 9, 2021
(86) PCT No.: PCT/EP2021/059354

§ 371 Date: Sep. 8, 2022

(30) Foreign Application Priority Data
Apr. 9, 2020 (GB) 2005321.1
Mar. 26, 2021 (EP) PCT/EP2021/057996

(51) Int. Cl. A61K 48/00; A61P 43/00

(52) U.S. Cl. CPC A61K 48/0066; A61P 43/00

(57) ABSTRACT

This disclosure concerns transcription cassettes comprising nucleic acid molecules comprising a nucleotide sequence encoding AP-4 subunits; vectors comprising said transcription cassettes; pharmaceutical compositions comprising said vector; and vectors or compositions for use in the treatment of AP-4-Hereditary Spastic Paraplegia.

### FIELD OF THE DISCLOSURE

This disclosure concerns transcription cassettes comprising nucleic acid molecules comprising a nucleotide sequence encoding at least one subunit of the heterotetrametric adaptor protein complex 4 (AP-4); vectors comprising said transcription cassettes; pharmaceutical compositions comprising said vector; and vectors or compositions for use in the treatment of AP-4 hereditary spastic paraplegias.

### BACKGROUND TO THE DISCLOSURE

Hereditary Spastic Paraplegias (HSPs) are a family of rare inherited, progressive, lower-limb spasticity disorders with an overall prevalence of 0.5-5.5 individuals per 100,000. Hereditary spastic paraplegia (HSP) in young patients is often characterised by weakness and spasticity (stiffness) of the legs and can later in life lead to further complications and may require the assistance of a cane, walker or wheelchair. There are a variety of genetic types of HSP such as autosomal dominant, autosomal recessive, X-linked, and maternally inherited (mitochondrial) forms with the autosomal dominant form most commonly found affecting between 75-80% of HSP patients. A variety of diagnostic methods for the identification of mutations in genes responsible for different forms of HSP, such as autosomal-recessive HSP (AR-HSP) caused by mutations in genes KIAA1840 (U.S. Ser. No. 10/519,503) or ZFYVE26 (US2017152562), or autosomal-dominant HSP caused by mutations in SPG3A, are disclosed in CN1958605.

AP-4-associated hereditary spastic paraplegia (AP-4-HSP), sometimes known as AP-4 deficiency syndrome or Adaptor protein complex 4 (AP-4) deficiency, is caused by loss-of-function mutations in any one of the four genes encoding the protein subunits of the AP-4 adaptor complex [10]. AP-4-HSP is autosomal recessive in nature. AP-4-HSP that is caused by mutations in the AP4B1 gene is sometimes called spastic paraplegia type 47 (SPG47) or hereditary spastic paraplegia 47 (HSP47) and it results in a significant decrease in AP4B1 protein levels [2]. AP-4-HSP may also be caused by mutations in the three other AP-4 subunits: AP4M1 mutations cause AP-4-HSP which is sometimes called SPG50 or HSP50, AP4E1 mutations cause AP-4-HSP which is sometimes called SPG51 or HSP51 and AP4S1 mutations cause AP-4-HSP which is sometimes called SPG52 or HSP51. AP-4-HSP characteristics are very similar regardless of the gene in which the causative mutations occur. The onset of AP-4-HSP usually occurs in early childhood and results in spasticity, intellectual disability from moderate to severe, impaired or absent speech, microencephaly, seizures, a shy character and in severe cases tetraplegia [11]. AP-4-HSP has so far been characterised in 199 children worldwide [1], however, incidents are most likely underreported. AP-4-HSP is progressive and there are no disease-modifying treatments. There is therefore a need to develop new therapies to improve patient outcomes for those suffering from AP-4-HSP.

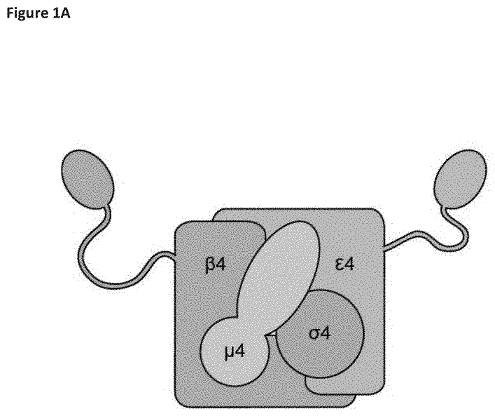

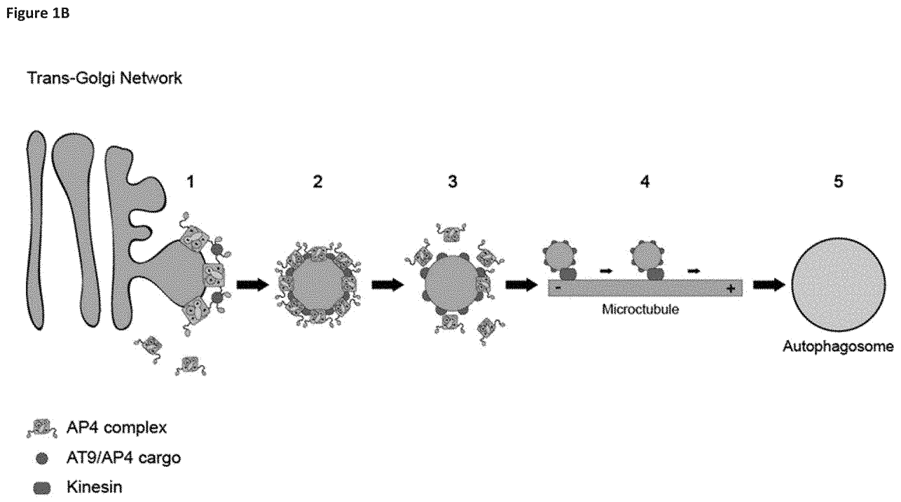

AP4B1 is one component of the AP-4 heterotetramer (FIG. 1A). The complete AP-4 complex is composed of two large adaptins (epsilon-type subunit AP4E1 and the beta-type subunit AP4B1), a medium adaptin (mu-type subunit AP4M1) and a small adaptin (sigma-type AP4S1). The AP-4 complex forms a non clathrin-associated coat on vesicles departing the trans-Golgi network (TGN) and may be involved in the targeting of proteins from the trans-Golgi network to the endosomal-lysosomal system (FIG. 1B). It is also involved in protein sorting to the basolateral membrane in epithelial cells and the proper asymmetric localization of proteins in neurons. AP-4 positive TGN derived vesicles are essential for correct spatial formation of autophagosomes and loss of the AP-4 complex can therefore impair autophagosome formation in the distal axon. Thus, the AP-4 complex is key to normal functioning in the brain.

Adeno-associated virus (AAV) vectors are known in the art and offer when compared to retroviral or lentiviral vectors a variety of advantages such as their mild immune response, capability to infect a broad range of cells and that the desired DNA is not integrated into the genome resulting in potential disruption and knock out of other genes but is stored extrachromosomal in the cell. AAV comprises single-stranded DNA genome of approximately 4.8 kilobases (kb) comprising three genes with coding sequences flanked by inverted repeats which are required for genome replication and packaging. Uses of AAVs and modified AAV vectors are known in the art and disclosed in WO2019/032898, WO2020041498 or WO2019/028306. AAV vectors have completed a variety of phase I and II clinical trials for the delivery of genes in the treatment of cystic fibroses and congestive heart failure and approved therapies for the treatment of spinal muscular atrophy.

We disclose expression vectors that include AP-4 nucleic acid molecules operably linked to expression control sequences adapted for expression in mammalian neurones, for example motor neurones, and the use of the modified expression vectors to deliver and functionally replace dysfunctional AP-4 proteins in the prevention or treatment of symptoms associated with HSPs. This disclosure relates to the development of modified vectors, for example AAV vectors, including nucleic acid molecules encoding proteins of the AP-4 complex.

### STATEMENTS OF INVENTION

According to an aspect of the invention there is provided an isolated nucleic acid molecule comprising: a transcription cassette comprising a promoter adapted for expression in a mammalian neurone said cassette further comprising a nucleic acid molecule comprising a nucleotide sequence that encodes at least one protein of the AP-4 complex.

In a preferred embodiment of the invention said expression cassette comprises a nucleic acid molecule comprising a nucleotide sequence selected from the group consisting of:
i) a nucleotide sequence, or polymorphic sequence variant, as set forth in SEQ ID NO:1 (AP4B1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 1 (AP4B1) wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 2 (AP4B1);
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (allows sequences that share at least 90%, 91%, 92%, 93%, 94%, 95%, 96%, 97%, 98% or 99% identity to hybridize)
Hybridization: 5×SSC at 65° C. for 16 hours
Wash twice: 2×SSC at room temperature (RT) for 15 minutes each
Wash twice: 0.5×SSC at 65° C. for 20 minutes each

High Stringency (allows sequences that share at least 80%, 81%, 82%, 83%, 84%, 85%, 86%, 87%, 88%, or 89% identity to hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each

Low Stringency (allows sequences that share at least 50%, 55%, 60%, 65%, 70% or 75% identity to hybridize)
Hybridization: 6×SSC at RT to 55° C. for 16-20 hours
Wash at least twice: 2×-3×SSC at RT to 55° C. for 20-30 minutes each.

In a preferred embodiment of the invention said expression cassette comprises a nucleic acid molecule comprising a nucleotide sequence selected from the group consisting of:
i) a nucleotide sequence, or polymorphic sequence variant, as set forth in SEQ ID NO:3 (AP4E1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 3 (AP4E1); wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 4 (AP4E1);
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

In a preferred embodiment of the invention said expression cassette comprises a nucleic acid molecule comprising a nucleotide sequence selected from the group consisting of:
i) a nucleotide sequence, or polymorphic sequence variant, as set forth in SEQ ID NO: 5 (AP4M1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 5 (AP4M1); wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 6 (AP4M1);
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

In a preferred embodiment of the invention said expression cassette comprises a nucleic acid molecule comprising a nucleotide sequence selected from the group consisting of:
i) a nucleotide sequence, or polymorphic sequence variant, as set forth in SEQ ID NO: 7 (AP4S1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 7 (AP4S1); wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 8 (AP4S1);
a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

In a preferred embodiment of the invention said cassette is adapted for expression in a motor neurone.

In a preferred embodiment of the invention said nucleic acid molecule comprises or consists of a nucleotide sequence as represented in SEQ ID NO: 1, or polymorphic sequence variant thereof.

In a preferred embodiment of the invention there is provided a nucleotide sequence or polymorphic sequence variant thereof, that encodes a polypeptide comprising an amino acid sequence as represented in SEQ ID NO: 2.

In a preferred embodiment of the invention said nucleic acid molecule comprises or consists of a nucleotide sequence as represented in SEQ ID NO: 3, or polymorphic sequence variant thereof.

In a preferred embodiment of the invention there is provided a nucleotide sequence or polymorphic sequence variant thereof that encodes a polypeptide comprising an amino acid sequence as represented in SEQ ID NO: 4.

In a preferred embodiment of the invention said nucleic acid molecule comprises or consists of a nucleotide sequence as represented in SEQ ID NO: 5, or polymorphic sequence variant thereof.

In a preferred embodiment of the invention there is provided a nucleotide sequence or polymorphic sequence variant thereof that encodes a polypeptide comprising an amino acid sequence as represented in SEQ ID NO: 6.

In a preferred embodiment of the invention said nucleic acid molecule comprises or consists of a nucleotide sequence as represented in SEQ ID NO: 7, or polymorphic sequence variant thereof.

In a preferred embodiment of the invention there is provided a nucleotide sequence or polymorphic sequence variant thereof, that encodes a polypeptide comprising an amino acid sequence as represented in SEQ ID NO: 8.

A polypeptide as herein disclosed may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies. In one embodiment, the polypeptides have at least 70% identity, even more preferably at least 75% identity, still more preferably at least 80%, 85%, 90%, 95% identity, and at least 99% identity the full-length amino acid sequence or nucleotide sequence illustrated herein.

In a preferred embodiment of the invention said promoter is a constitutive promoter.

In an alternative embodiment of the invention said promoter is a regulated promoter, for example an inducible or cell specific promoter.

In a preferred embodiment of the invention said promotor is selected from the group consisting of: chicken beta actin (CBA) promoter, chicken beta actin hybrid (CBh) promoter, CAG promoter, JeT promoter, neuronal and glial specific promoters including synapsin 1, Hb9, MeP229 and GFAP promoter sequences, as well as AP-4 subunit specific promoter regions including AP4B1, AP4E1, AP4M1 and AP4S1.

In a preferred embodiment of the invention said promoter sequence comprises a nucleic acid comprising a nucleotide sequence as set forth in SEQ ID NO: 27, or a nucleotide sequence that is a polymorphic sequence variant of SEQ ID NO: 27.

In an alternative embodiment of the invention said promoter sequence comprises a nucleic acid comprising a nucleotide sequence as set forth in SEQ ID NO: 30, or a nucleotide sequence that is a polymorphic sequence variant of SEQ ID NO: 30.

According to a further aspect of the invention there is provided transcription cassette comprising:
a first nucleic acid molecule comprising a nucleotide sequence as set forth in SEQ ID NO: 27, or a nucleotide sequence that is a polymorphic sequence variant of SEQ ID NO: 27, wherein said nucleic acid molecule is a transcription promoter and operably linked to a second nucleic acid molecule comprising a nucleotide sequence encoding a polypeptide wherein the first nucleic acid molecule regulates transcription of the second nucleic acid molecule.

In a preferred embodiment of the invention said first nucleic acid molecule comprising a promoter comprises or consists of the nucleotide sequence set forth in SEQ ID NO: 27.

In an alternative embodiment of the invention said first nucleic acid molecule comprises a nucleotide sequence as set forth in SEQ ID NO: 30, or a nucleotide sequence that is a polymorphic sequence variant of SEQ ID NO: 30.

In a preferred embodiment of the invention said second nucleic acid molecule comprises a nucleotide sequence that encodes at least one polypeptide of the AP-4 complex.

In a preferred embodiment of the invention said second nucleic acid molecule comprises or consists of a nucleotide sequence as represented in SEQ ID NO: 1, or polymorphic sequence variant thereof.

In a preferred embodiment of the invention there is provided a nucleotide sequence or polymorphic sequence variant thereof, that encodes a polypeptide comprising an amino acid sequence as represented in SEQ ID NO: 2.

In a preferred embodiment of the invention said nucleic acid molecule comprises or consists of a nucleotide sequence as represented in SEQ ID NO: 3, or polymorphic sequence variant thereof.

In a preferred embodiment of the invention there is provided a nucleotide sequence or polymorphic sequence variant thereof, that encodes a polypeptide comprising an amino acid sequence as represented in SEQ ID NO: 4.

In a preferred embodiment of the invention said nucleic acid molecule comprises or consists of a nucleotide sequence as represented in SEQ ID NO: 5, or polymorphic sequence variant thereof.

In a preferred embodiment of the invention there is provided a nucleotide sequence or polymorphic sequence variant thereof, that encodes a polypeptide comprising an amino acid sequence as represented in SEQ ID NO: 6.

In a preferred embodiment of the invention said nucleic acid molecule comprises or consists of a nucleotide sequence as represented in SEQ ID NO: 7, or polymorphic sequence variant thereof.

In a preferred embodiment of the invention there is provided a nucleotide sequence or polymorphic sequence variant thereof, that encodes a polypeptide comprising an amino acid sequence as represented in SEQ ID NO: 8.

“Promoter” or “transcription promoter” are art recognised and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to several physiological/environmental cues and can be constitutive or regulatable and also cell/tissue specific. Promoter elements also include so called TATA box and RNA polymerase initiation selection (RIS) sequences which function to select a site of transcription initiation. These sequences also bind polypeptides which function, inter alia, to facilitate transcription initiation selection by RNA polymerase.

As used herein, first nucleic acid comprising a promoter sequence and second nucleotide sequence encoding a polypeptide are said to be “operably” linked when they are covalently linked in such a way as to place the expression or transcription of the second nucleic acid molecule under the control of the first nucleic acid molecule comprising regulatory sequences. If it is desired that the coding sequences be translated into a functional protein, two DNA sequences are said to be operably linked if induction of a promoter in the 5′ regulatory sequences result in the transcription of the coding sequence and production of mRNA. Thus, a promoter region would be operably linked to a coding sequence if the promoter region were capable of effecting transcription of that DNA sequence such that the resulting transcript IS translated into the desired protein or polypeptide.

According to a further aspect of the invention there is provided an expression vector comprising a transcription cassette according to the invention.

Viruses are commonly used as vectors for the delivery of exogenous genes. Commonly employed vectors include recombinantly modified enveloped or non-enveloped DNA and RNA viruses, for example baculoviridiae, parvoviridiae, picornoviridiae, herpesveridiae, poxviridae, adenoviridiae, picornnaviridiae or retroviridae e.g. lentivirus. Chimeric vectors may also be employed which exploit advantageous elements of each of the parent vector properties (See e.g., Feng, et al (1997) Nature Biotechnology 15:866-870). Such viral vectors may be wild-type or may be modified by recombinant DNA techniques to be replication deficient, conditionally replicating or replication competent. Conditionally replicating viral vectors are used to achieve selective expression in particular cell types while avoiding untoward broad-spectrum infection. Examples of conditionally replicating vectors are described in Pennisi, E. (1996) Science 274:342-343; Russell, and S. J. (1994) Eur. J. of Cancer 30A(8):1165-1171.

Preferred vectors are derived from the adenoviral, adeno-associated viral or retroviral genomes.

In a preferred embodiment of the invention said expression vector is a viral based expression vector.

In a preferred embodiment of the invention said viral based vector is an adeno-associated virus [AAV].

In a preferred embodiment said viral based vector is selected from the group consisting of: AAV2, AAV3, AAV6, AAV13; AAV1, AAV4, AAV5, AAV6, AAV9 and rhAAV10.

In a preferred embodiment of the invention said viral based vector is AAV9.

In a preferred embodiment of the invention said AAV vector is based on a single stranded AAV virus.

In an alternative embodiment of the invention said AAV vector is based on a self-complementary AAV virus.

Naturally occurring AAV serotypes typically comprise a single stranded genome which during natural infection is replicated to form a double stranded AAV viral genome. This is a rate limiting step in AAV replication and expression. A recombinant form of AAV is referred to as self-complementary AAV which comprise both a sense and antisense genomic strands that are adapted for immediate expression and replication.

In a preferred embodiment of the invention said viral based vector comprises the nucleotide sequence set forth in SEQ ID NO: 19 (AP4B1).

In a preferred embodiment of the invention said viral based vector comprises the nucleotide sequence set forth in SEQ ID NO: 20 (AP4B1).

In a preferred embodiment of the invention said viral based vector comprises the nucleotide sequence set forth in SEQ ID NO: 21 (AP4S1).

In a preferred embodiment of the invention said viral based vector comprises the nucleotide sequence set forth in SEQ ID NO: 22 (AP4S1).

In a preferred embodiment of the invention said viral based vector comprises the nucleotide sequence set forth in SEQ ID NO: 23 (AP4E1).

In a preferred embodiment of the invention said viral based vector comprises the nucleotide sequence set forth in SEQ ID NO: 24 (AP4E1).

In a preferred embodiment of the invention said viral based vector comprises further SEQ ID NO 25 or 26.

In an alternative preferred embodiment of the invention said viral based vector is a lentiviral vector.

According to a further aspect of the invention there is provided a pharmaceutical composition comprising an expression vector according to the invention and an excipient or carrier.

The expression vector compositions of the present invention are administered in pharmaceutically acceptable preparations. Such preparations may routinely contain pharmaceutically acceptable concentrations of salt, buffering agents, preservatives, compatible carriers and supplementary therapeutic agents. The expression vector compositions of the invention can be administered by any conventional route, including injection or by gradual infusion over time.

The expression vector compositions of the invention are administered in effective amounts. An “effective amount” is that amount of the expression vector that alone, or together with further doses, produces the desired response. In the case of treating a disease, the desired response is inhibiting the progression of the disease. This may involve only slowing the progression of the disease temporarily, although more preferably, it involves halting the progression of the disease permanently. This can be monitored by routine methods. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner.

These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The expression vector compositions used in the foregoing methods preferably are sterile and contain an effective amount of expression vector according to the invention for producing the desired response in a unit of weight or volume suitable for administration to a patient. The doses of vector administered to a subject can be chosen in accordance with different parameters, in particular in accordance with the mode of administration used and the state of the subject. Other factors include the desired period of treatment. If a response in a subject is insufficient at the initial doses applied, higher doses (or effectively higher doses by a different, more localized delivery route) may be employed to the extent that patient tolerance permits. Other protocols for the administration of vector compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the expression vector compositions of the invention are applied in pharmaceutically acceptable amounts and in pharmaceutically acceptable compositions. The term “pharmaceutically acceptable” means a non-toxic material that does not interfere with the effectiveness of the biological activity of the active agent. Such preparations may routinely contain salts, buffering agents, preservatives, compatible carriers, and optionally other therapeutic agents' (e.g., those typically used in the treatment of the specific disease indication). When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

The pharmaceutical compositions containing the expression vectors according to the invention may contain suitable buffering agents, including acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The expression vector compositions may conveniently be presented in unit dosage form and may be prepared by any of the methods well-known in the art of pharmacy. All methods include the step of bringing the active agent into association with a vector which constitutes one or more accessory ingredients. The preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1, 3-butanediol. Among the acceptable solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical

Sciences, Mack Publishing Co., Easton, Pa.

According to a further aspect of the invention there is provided an expression vector according to the invention for use as a medicament.

According to a further aspect of the invention there is provided an expression vector according to the invention for use in the treatment of AP-4 Hereditary Spastic Paraplegias.

In a preferred embodiment of the invention said AP-4-HSP is SPG47 In a preferred embodiment of the invention said AP-4-HSP is SPG50.

In a preferred embodiment of the invention said AP-4-HSP is SPG51.

In a preferred embodiment of the invention said AP-4-HSP is SPG52.

Spastic Paraplegia (SPG) is used interchangeably with Hereditary Spastic Paraplegia (HSP), thus SPG47 is HSP47, SPG50 is HSP50, SPG51 is HSP51 and SPG52 is HSP52.

According to a further aspect of the invention there is provided a cell transfected with an expression vector according to the invention.

In a preferred embodiment of the invention said cell is a neurone.

In a preferred embodiment of the invention said neurone is a motor neurone.

According to a further aspect of the invention there is provided a method to treat or prevent AP-4 Hereditary Spastic Paraplegias comprising administering a therapeutically effective amount of an expression vector according to the invention to prevent and/or treat Hereditary Spastic Paraplegias.

In a preferred method of the invention said AP-4-HSP is Spastic Paraplegia type 47 (SPG47).

In a preferred method of the invention said AP-4 HSP is Spastic Paraplegia type 50 (SPG50). In a preferred method of the invention said AP-4 HSP is Spastic Paraplegia type 51 (SPG51).

In a preferred method of the invention said AP-4 HSP is Spastic Paraplegia type 52 (SPG52).

According to a further aspect of the invention there is provided a diagnostic method to genotype a subject to determine whether the subject has a mutation in one or more AP-4 gene sequences comprising the steps:
i) obtaining a biological sample from a subject to be tested and extracting nucleic acid from said biological sample;
ii) sequencing the nucleic acid to obtain a nucleotide sequence of AP4B1, AP4E1, AP4M1 or AP4S1 in said subject;
iii) comparing the obtained genomic sequence with a normal matched control nucleotide sequence to identify nucleotide sequence differences; and
iv) determining whether the test sample is modified in an AP-4 gene sequence and whether said modification is associated with a Hereditary Spastic Paraplegias type.

In a preferred method of the invention said method further comprises the administration of at least one expression vector according to the invention to prevent or treat a type of AP-4 Hereditary Spastic Paraplegias.

In a preferred method of the invention said AP-4-HSP selected from the group consisting of: SPG47, SPG50, SPG51 and SPG52. In a preferred method of the invention said genomic sequence comprises SEQ ID NO: 1, SEQ ID NO: 3, SEQ ID NO: 5 or SEQ ID NO: 7, or a polymorphic sequence variant thereof.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

FIG. 1—The AP4 complex and function: (A) Illustration of the AP4 heterotetramer complex, comprised of the large beta and epsilon-type adaptins (β4 and ε4, termed AP4B1 and AP4E1), the medium mu-type adaptin (APμ4, termed AP4M1) and the small sigma adaptin (APσ4, termed AP4S1). (B) Illustration of the AP4 complex function 1) AP4 heterotetramers are recruited to the trans-Golgi network (TGN) and in turn recruit their cargo proteins, including ATG9. 2) clatherin negative vesicles bud from the TGN. 3) The AP4 complex is shed from the vesicles and recycled back to the TGN for further vesicle formation. 4) Remaining vesicles are bound by Kinesin motor proteins and transported in an anterograde direction along microtubules to the cell periphery or distal neuronal compartments. 5) ATG9 vesicles assemble to promote autophagosome formation;

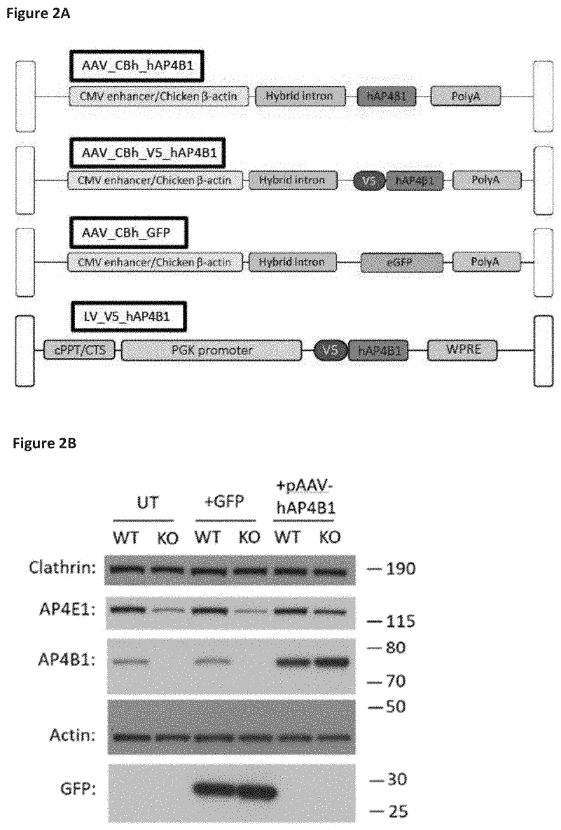

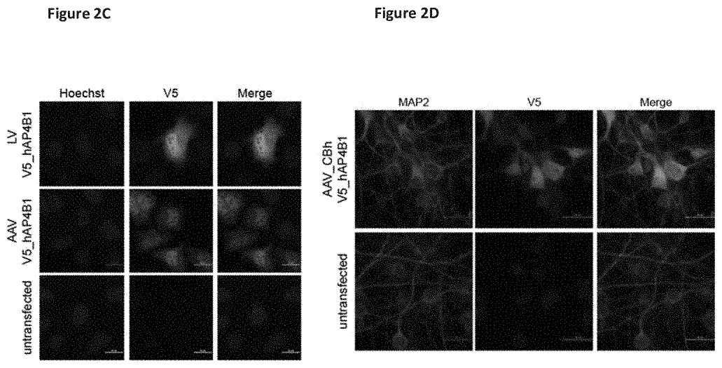

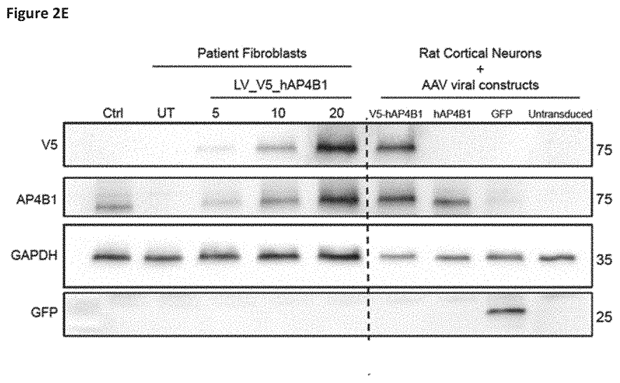

FIG. 2—Design and validation of gene therapy vectors for AP4B1 gene replacement. (A) Schematic illustration of the AAV and LV designed and already in place. (B) Representative western blot of control (WT) or AP4B1-knockout (KO) HeLa cell lysates after transfection with plasmids expressing GFP (+GFP) or hAP4B1 (+AAV-hAP4B1). Expression of hAP4B1 in KO cell lines rescues missing AP4B1 protein expression. Rescue of AP4B1 expression also restores expression of AP4E1 subunit protein levels to WT levels. (C) Validation of AAV-expressing V5 tagged AP4B1 in AP4B1−/− Hela cells. (D) Non-transgenic rat cortical neurons stained with cortical neuron marker MAP2 (Red channel) and V5 (Green channel) primary antibodies, 10 days post-treatment with either 300,000 vg/cell AAV9-V5_hAP4B1. (E) Representative western blot of non-disease control fibroblasts (Ctrl), and SPG47 patient fibroblasts both untreated (UT) and treated with increasing amounts of LV-V5_hAP4B1 showing rescue of hAP4B1 expression in patient mutant lines (left side of dashed line). Representative western blot of non-transgenic rat cortical neurons treated with 400,000 vg/cell AAV9 viral vectors: AAV9-V5_SPG47(hAP4B1); AAV9-SPG47(hAP4B1); AAV9-GFP; non-transduced cells;

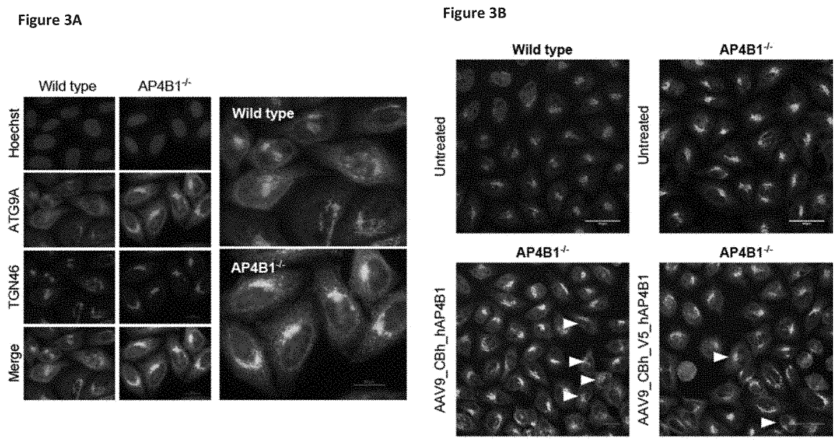

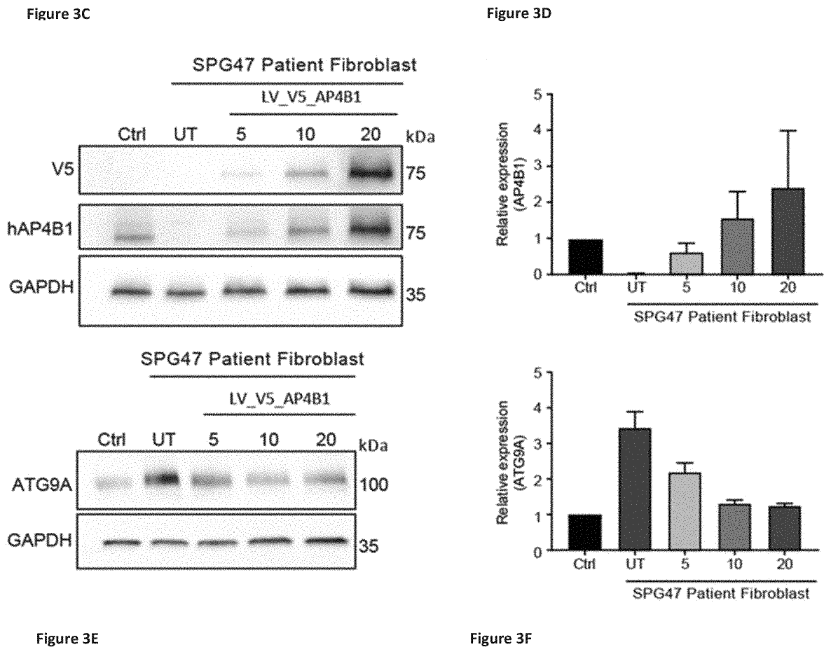

FIG. 3—Use of ATG9A as readout to assess efficacy of AB4B1 gene replacement. (A) illustration of the mislocalisation of ATG9A to the TGN trans-golgi network (TGN) in CRISPR generated Hela knockout cell model. ATG9A mislocalisation in AP4B1−/− Hela cells is rescued after transfection with AAV9 constructs encoding AP4B1 (B). Representative western blot of LV transduced patient cells shows rescue of hAP4B1 expression and detection of V5 tag (C), quantified in (D). Transduced patient cells also show rescue of ATG9A expression (E), quantified in (F). Data is presented as mean+/−standard error of the mean (SEM), n=3. Data analysed by one-way ANOVA followed by post-hoc Dunnett's multiple comparisons test with respect to Ctrl. Stars indicate p 0.05 (*); p<0.0001 (****); ns=not significant. (G) Lentiviral vector (LV)-mediated correction of ATG9A mislocalisation in SPG47 patient primary fibroblast cells. SPG47 patient cells marked with white asterisks show rescue of mislocalised ATG9A after treatment with LV-V5_hAP4B1;

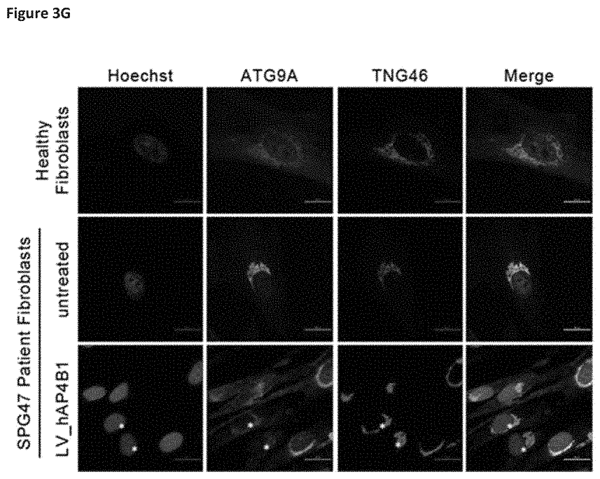

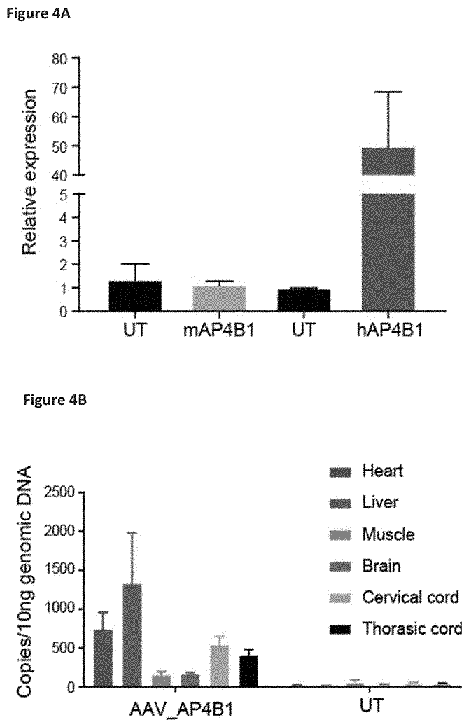

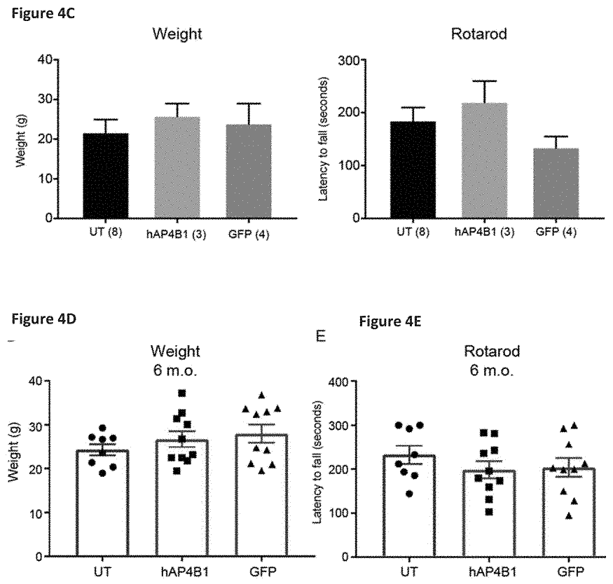

FIG. 4—(A) Quantification of mouse Ap4b1 (pink) and human AP4B1 (green) mRNA by RT-qPCR in brain tissue, 4-weeks post-injection via cisterna magna of AAV9_CBh_hAP4B1 (UT—untreated, n=3; AAV_CBh_hAP4B1, n=5). (B) Quantification of viral genome copy number by qPCR in various CNS and peripheral tissues, 4-weeks post-injection via cisterna magna of 2×10¹⁰ vg AAV9_CBh_hAP4B1 viral vector (UT—untreated, n=3; AAV_CBh_hAP4B1, n=5). (C) Pilot safety in vivo study in wild type mice. Weight and rotarod measurements of wildtype mice at 4 weeks post-injection via cisterna magna of 2×10¹⁰ vg AAV_CBh_hAP4B1 or AAV_CBh_GFP viral vectors. N number for each group shown in parentheses. (D & E) Pilot safety in vivo study in wild type mice. Weight and rotarod measurements of wildtype mice at 6 months post-injection via cisterna magna of 2×10¹⁰ vg AAV_CBh_hAP4B1 or AAV_CBh_GFP viral vectors. N=8 (UT), 10 (hAP4B1/GFP);

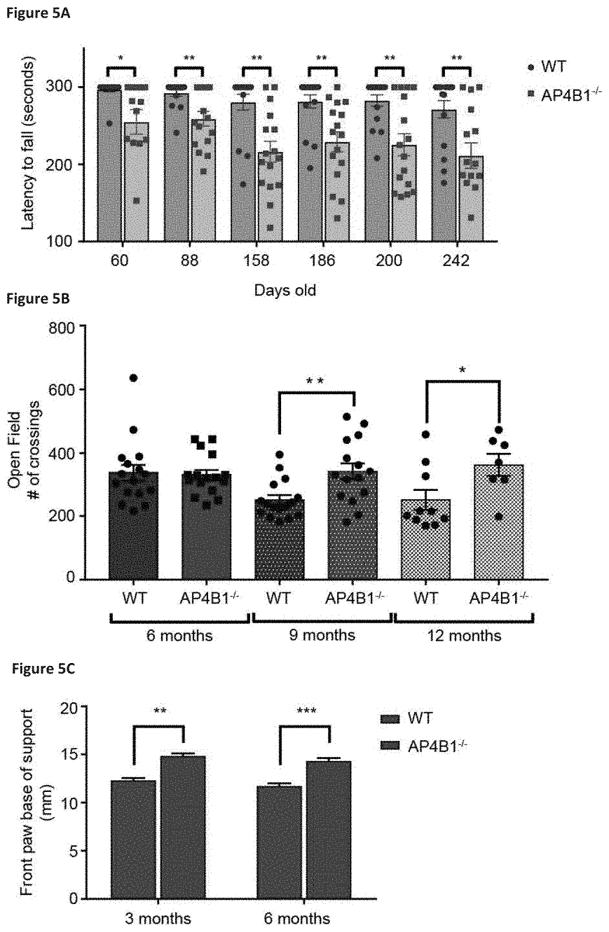

FIG. 5—Characterisation of Ap4b1^{-/-} mouse model. Histogram showing rotarod (A) assessment of wildtype (WT) and Ap4b1-knockout (Ap4b1^{-/-}) mice displayed as latency to fall in seconds, at various. Data is presented as mean+/−standard error of the mean (SEM), n=16 per group except Ap4b1 (−/−) Day 242 where n=15. Data analysed by Student's t-test with respect to WT. Stars indicate p 0.05 (*); p<0.01 (**). (B) Open-field behavioural assessment of Ap4b1-knockout (Ap4b1^{-/-})) compared to wildtype (WT) mice at 6, 9 and 12-months of age. Data is presented as mean+/−standard error of the mean (SEM), n is variable for all groups and timepoints as study is ongoing. Data analysed by One-way ANOVA with each group compared to every other group followed by Tukey's post-hoc multiple comparisons test. Stars indicate p 0.05 (*). (C) Front paw base of support as measured by CatWalk assessment at 3 months and 6 months of age. Data analysed by Two-way ANOVA with Sidak's post-hoc multiple comparisons test. Stars indicate p<0.01 (**); p<0.001 (***); (D) Gait analysis

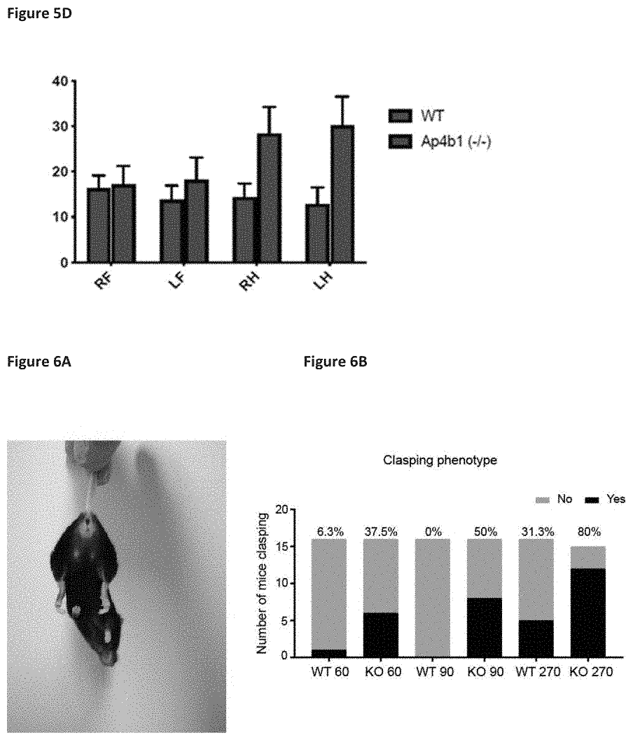

FIG. 6—(A) Image demonstrating clasping phenotype observed in Ap4b1 (−/−) mice, with hindlimbs drawn inwards towards the midline. (B) Histogram showing presence of clasping phenotype in wildtype (WT) and Ap4b1-knockout (Ap4b1^{-/-}) at different timepoints. Data is presented as number of mice in each group that do or do not demonstrate clasping phenotype after suspension by the tail. The percentage of mice in each group positive for hindlimb clasping is shown above. n=16 per group except Ap4b1 (−/−) Day 270 where n=15;

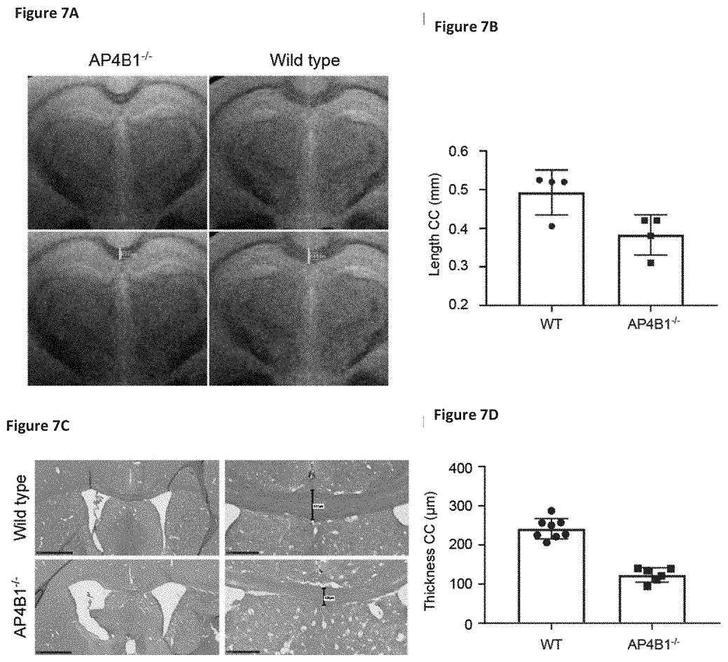

FIG. 7—Coronal MRI images show a reduction in corpus callosum thickness in Ap4b1-ko (Ap4b1^{-/-}) mouse compared to a wildtype mouse (A). Thickness in μm is quantified in (B). Preliminary data presented as mean+/−SD. Measurements are from 4 slices per mouse taken at matching brain regions. Representative images from coronal brain sections stained with H&E confirming the MRI findings (C). Measurements of corpus collosum thickness are from multiple slices per mouse taken at matching brain regions. Thickness in μm is quantified in (D). Data presented as mean+/−SD. Scale bars=1 mm; 250 μm. Imaged and analysed using the NanoZoomer Digital Pathology software (Hamamatsu);

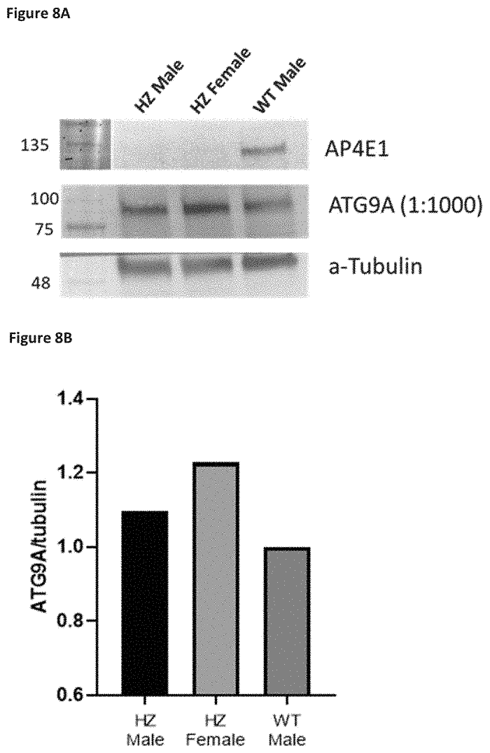

FIG. 8. Status of AP4 complex in AP4B1 knockout mice ATG9A and AP4E1 protein levels were assessed in the brain collected from homozygote Ap4b1−/− (HZ) and wild type (WT) mice. Increased ATG9A levels in Ap4b1−/− in comparison to WT mice. This is a phenotype observed in human cell models including human patient fibroblasts and iPSCs derived neurons. Another important observation is the depletion of AP4E1 unit of the AP4 complex in AP4b1−/− when compared to WT mice. The same observation has been reported in cell model systems of SPG47 including human cells isolated from AP4 patients;

FIG. 9. Weight gain in AAV9-hAP4B1 treated Ap4b1−/− mice. Group average for untreated wildtype mice (Black); untreated AP4b1−/− mice (blue) and Ap4B1−/− mice treated by intra cisterna magna injection of 5×1013 gc/kg of AAV9-hAP4B1 on postnatal day1 (red). Female and male are plotted separately;

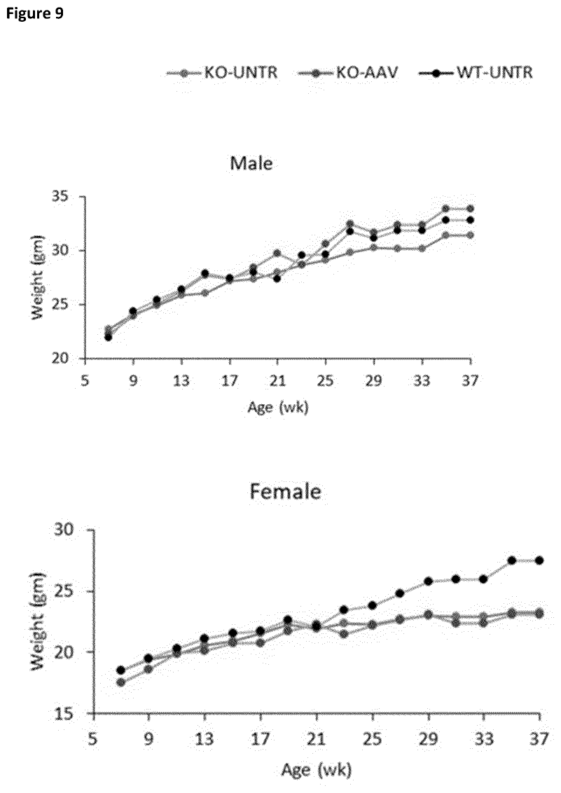

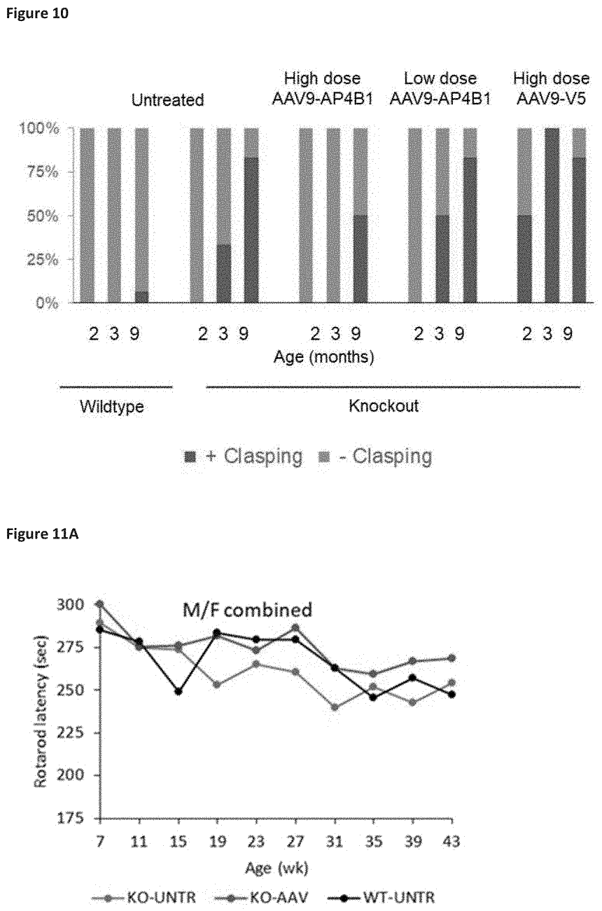

FIG. 10. AAV9-hAP4B1 Treatment response by clasping assay. Cohorts varying from n=6 to n=15. Either wildtype or Ap4b1−/− knockout mice were assessed for clasping reflex at ages of 2, 3 and 9 months and % positive for clasping is plotted (red). Low dose=2×1013 gc/kg; High dose=5×1013 gc/kg;

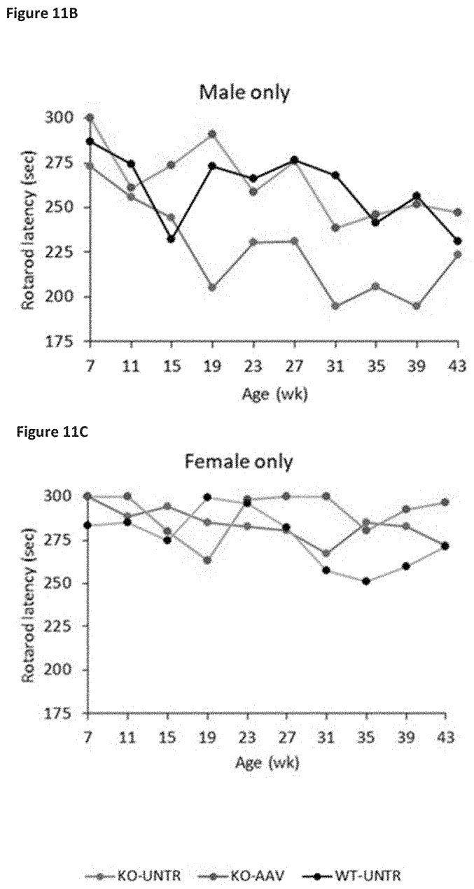

FIG. 11. Impact of intra cisterna magna administration of AAV9-hAP4B1 on latency in rotarod test. Group average for untreated wildtype mice (Black); untreated AP4b1−/− mice (blue) and Ap4B1−/− mice treated by intra cisterna magna injection of 5×1013 gc/kg of AAV9-hAP4B1 on postnatal day1 (red). Female and male are plotted separately;

FIG. 12. Status of AP4 complex in AP4B1 knockout mice. AP4E1 protein levels were restored in the spinal cord (A) and Heart (B) collected from homozygote Ap4b1−/− (HZ) and wild type (WT) mice;

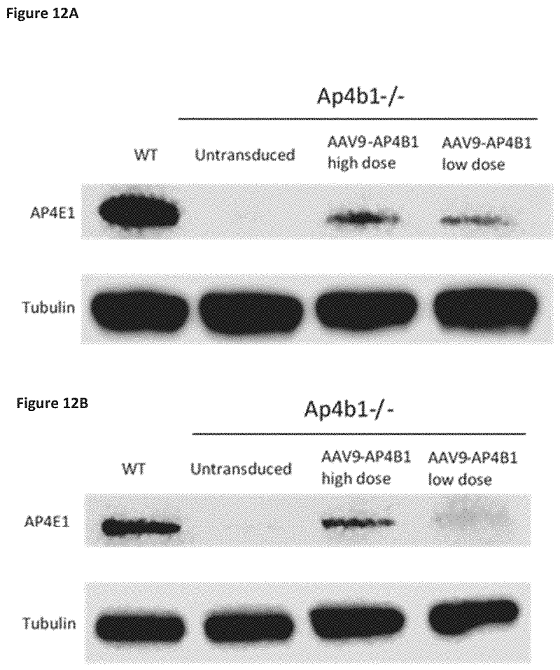

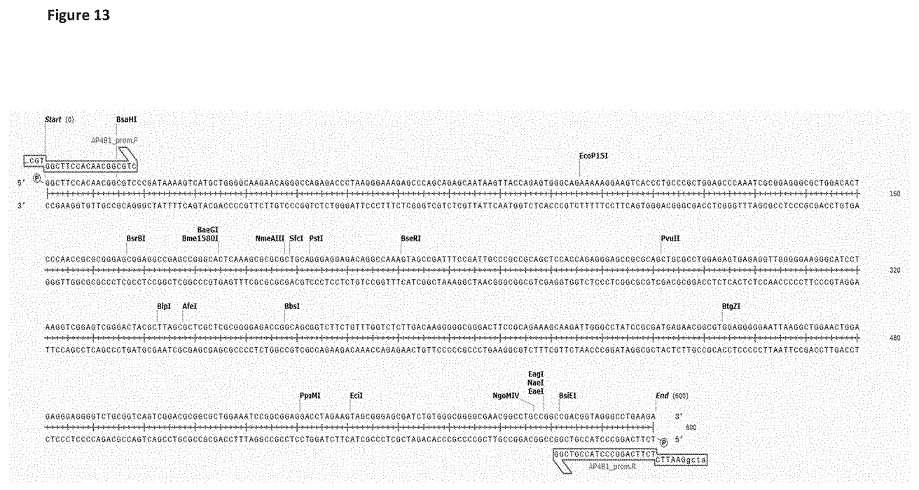

FIG. 13. describes AP4B1 endogenous promoter sequence and primers;

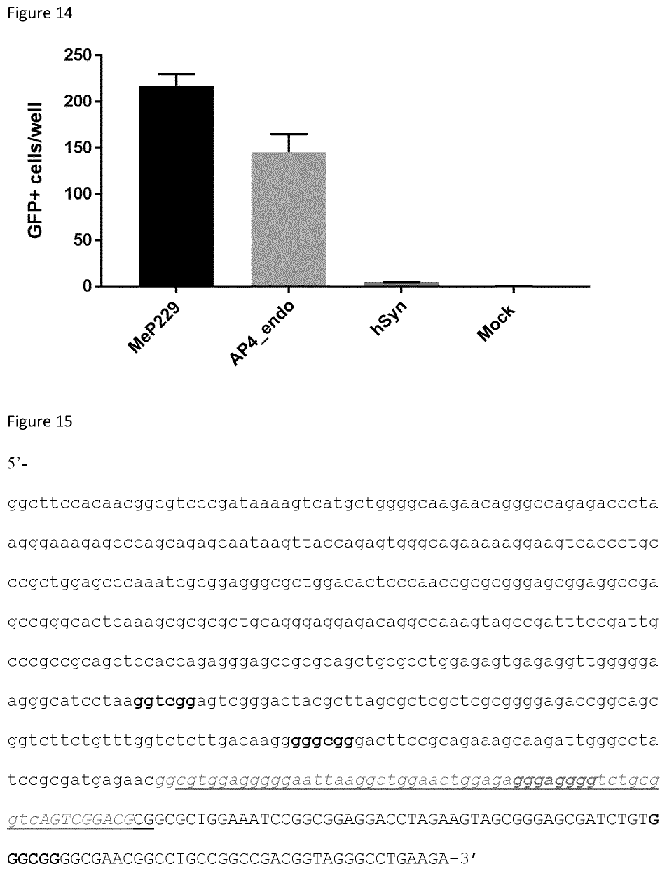

FIG. 14. GFP expression in HeLa cells under the control of the MeP229, AP4 and hSyn promoters. The term “mock” indicates the control with no expression of GFP. As shown, expression under all three promoters was detected. Experiment was performed using three replicas for each sample. Data presented as mean+SD.

FIG. 15. Annotated sequence showing the TSS position (uppercase) and minimal promoter region (Underlined). GC-Box regions often found in promoters and regulatory sequence are in bold. Further highlighted is the PoIII chip-seq target region (italics).

### Materials and Methods

### Ethics Statement

All animal in vivo experiments were approved by the University of Sheffield Ethical Review Sub-Committee, the UK Animal Procedures Committee (London, UK) and performed according to the Animal (Scientific Procedures) Act 1986, under the Project License 40/3739. C57BL/6J-Ap4b1^{em5Lutzy}/J mice and non-transgenic C57BLJ6J mice were maintained in a controlled facility in a 12 h dark/12 h light photocycle (on at 7 am/off at 7 μm) with free access to food and water. The ARRIVE guidelines have been followed in reporting this study.

### Viral Vector Construction

The original pAAV2 vector backbone was published in Laughlin et al., (1983). The CBh promoter⁴—including the hybrid intron region—was amplified by PCR from the pTRS-KS-CBh-eGFP plasmid provided by Dr S. Gray and cloned into the MluI and EcoRI sites of the pAAV_CMV_MCS construct after removal of the CMV promoter by restriction digest. Full length hAP4B1 cDNA was transferred to pAAV-CBh-MCS by PCR amplification and ligation from the pLXIN-SPG47 plasmid provided by Dr J. Hirst⁵. The hAP4B1 cDNA sequence was cloned between the SalI and HindIII sites of the pAAV_CBh_MCS plasmid. A separate epitope-tagged construct (pAAV_CBh_V5-hAP4B1) was created to enable detection of hAP4B1 protein expression by ICC. A linkerless N-terminal V5 epitope tag was inserted immediately downstream of the hAP4B1 Kozak sequence by Q5 mediated site-directed mutagenesis using a back-to-back primer strategy designed for large insertions. For investigating gene transfer in human fibroblast cell lines, the V5-hAP4B1 transgene sequence was subcloned by restriction digest (SalI/NotI 4 XhoI/NotI) into the pLenti_PGK_MCS_Vos lentiviral backbone (provided by Dr K. de Vos), downstream of the PGK promoter.

pCMV3-AP4S1 was purchased from Sino Biological. Full-length human untagged AP4S1 was transferred to pAAV_CBh_MCS by PCR amplification from pCMV3-AP4S1 to introduce AgeI and XbaI restriction sites on the 5′ and 3′ ends of hAP4S1 respectively. The hAP4S1 cDNA product was then cloned between the AgeI and XbaI sites of the pAAV_CBh_MCS plasmid. In addition to constructs driving gene expression using the strong constitutive CBh promoter, three further putative expression vectors were generated containing weaker promoters that were hypothesised to express the hAP4B1 transgene at a level closer to that found endogenously. The promoters were MeP229 (derived from a core fragment of the endogenous Mecp2 gene promoter, amplified by PCR from the pSJG-MeP229-GFP plasmid received as a gift from Dr S. Gray), hSyn (neuronal cell-specific human synapsin 1 gene promoter, cloned by restriction digest-ligation from scAAV-SYN1-GFP, a construct originally published by Lukashchuk and colleagues⁶) and AP4B1_endo (a putative endogenous promoter containing the region ˜600 bp upstream of the hAP4B1 gene transcription start site. This region was identified in silico as a potential promoter region due its genomic location, presence of a CpG island and presence of a CCCTC-binding factor (CTCF) binding region—a known transcriptional activator⁷.) All three promoters were cloned into pAAV_MCS_KanR between the MluI and EcoRI sites by restriction ligation. AP4B1_endo was amplified by PCR from human genomic DNA extracted from HEK293 cells.

To serve as a negative control for the V5-tagged hAP4B1 construct, the V5 epitope alone was PCR amplified from the pCIneo_V5-N vector (gift from Dr K. de Vos) and cloned into the XbaI site of pAAV_CBh_MCS. The eGFP transgene from pTRS-KS-CBh-GFP was cloned into the pAAV_CBh_MCS vector by restriction digest between the AgeI/BamHI sites to generate a GFP expression control vector (pAAV_CBh_eGFP).

### Initial Safety Study—Cisterna Magna Delivery of Viral Gene Therapy Constructs in P1 Mice.

Post-natal day 1 (P1) wildtype C57BI/6J mice were anaesthetised by isoflurane. Induction occurred in a chamber at 5% isoflurane, 3 L O2/minute. Anaesthesia was maintained via a mask at 1-2% isoflurane, 0.3 L O2/minute for approximately 5 minutes during injection. The cisterna magna was located using a Wee-Sight transilluminator vein finder (Phillips). Viral vectors (pAAV_CBh_hAP4B1 and pAAV_CBh_eGFP) were injected directly into the cisterna magna of P1 mice (n=15 per group), using stereotaxic apparatus containing a 33-gauge Hamilton syringe with automated perfusion pump. The solution was administered at a flow-rate of 1 μL per minute; the maximum volume of solution administered was 5 μL per animal. Animals each received a maximum dose of 5×10¹⁰ total vector genomes. The experimental timeline proceeded as follows:

Day 1—Postnatal day 0, day of birth (P0)—Footpad tattoos applied for identification purposes Day 2—Postnatal day 1 (P1)—Injection of up to 5 μL of viral vector or vehicle solution into the cisterna magna, under isoflurane anaesthesia.

Day 29 (or Day 170)—Postnatal day 28 (P28) or P168 (6 months post-injection)—Animals were perfused under terminal anaesthesia, and tissue samples collected for analysis.

### Cisterna Magna Delivery of Viral Gene Therapy Constructs in P1 Mice as Proof-of-Concept.

A study to assess the ability of our therapeutic viral vector (pAAV_CBh_hAP4B1) to mediate transgene expression in the central nervous system (CNS) of transgenic mice lacking endogenous Ap4b1 (KO C57BLJ6J-Ap4b1^{em5Lutzy}/J) after injection via the cisterna magna, was followed. Mice were injected via the cisterna magna as in the previously described safety study. Two viral vectors were used; an AAV9 expressing a full length copy of the human AP4B1 (SPG47) gene and, an AAV9 expressing a V5 tag with no additional coding sequence as a viral control. Mice receiving AAV9-hAP4B1 viral vector were injected with two different doses (a low dose of 2×10¹⁰ vector genomes and a high dose of 4×10¹⁰ vector genomes, respectively), whereas mice receiving AAV9-V5 were injected with a high dose (4×10¹⁰ vector genomes) only. Two more groups were included in the study; untreated KO C57BL/6J-Ap4b1^{em5Lutzy}/J and untreated WT C57BL/6J-Ap4b1^{em5Lutzy}/J. Rescue of the phenotype was assessed by improvements in behavioural parameters, that will be described in details below, in the treated mice compared with untreated.

### Genotyping and Colony Maintenance

C57BL/6J-Ap4b1^{em5Lutzy}/J mice were generated by Jackson Labs using CRISPR-Cas9 mediated deletion of a 76 bp region within Exon 1 of the murine Ap4b1 gene. Deletion of this region generated a frameshift mutation and a truncated mRNA transcript. WT Sequence (deletions in lower case):

(SEQ ID NO 9)
TTGGCGACGATGCCATAccttggctctgaggacgtggtgaaggaactgaa
gaaggctctgtgtaaccctcatattcaggctgataggctgcgcTACCGGA
ATGTCATCCAGCGAGTTATTAGGTATCACCAACCTACCATAGAA.

Genotyping of mice was performed based on the protocol optimised by Charles River Laboratories. Mouse genotyping was performed on genomic DNA extracted from tail or ear tissue by the addition of 20 μl QuickExtract™ DNA Extraction Solution (Lucigen) and incubation on a thermocycler for 15 minutes at 65° C. followed by 2 minutes at 98° C. Genotyping PCRs were performed in a 20 μl volume reaction as separate reactions for WT and KO alleles. Reactions consisted of 5 μl 5× FIREPol® Master Mix Ready to Load with 7.5 mM MgCl₂ (Solis Biodyne), 500 nM each of genotyping primers—P1+P2 for WT allele amplification and P1+P3 for KO allele amplification—(P1: 5′-TCGCCCGAGGACCCAAGAA-3′(SEQ ID NO 10); P2: 5′-CCTATCAGCCTGAATATGAGGGTTACA-3′ (SEQ ID NO 11); P3: 5′-GCTGGATGACATTCCGGTATATG-3′ (SEQ ID NO 12)) and 1 μl genomic DNA from the QuickExtract™ protocol. Touchdown PCR was performed according to the thermal profile shown in Table 1. Following PCR and agarose gel electrophoresis (2% agarose gel in Tris-acetate-EDTA buffer), WT and KO allele PCR products were visualised at ˜254 bp and ˜203 bp respectively.

Heterozygous mice were bred together to produce homozygous WT (Ap4b1+/+), KO (Ap4b1−/−) and heterozygous (Ap4b1+/−) littermates.

TABLE 1
Touchdown PCR conditions for C57BL/6J-Ap4b1^{em5Lutzy}/J genotyping
PCS conditions

| step | temp [° C.] | time [sec] | |
|---|---|---|---|
| 1) Initial denaturation | 94 | 120 | |
| 2) Denaturation | 94 | 60 | Cycle no. (2-4) × 10 |
| 3) Annealing | TD 58-54 | 60 | |
| 4) Elongation | 72 | 60 | |
| 5) Denaturation | 94 | 60 | Cycle no. (5-7) × 25 |
| 6) Annealing | 54 | 60 | |
| 7) Elongation | 72 | 60 | |
| 8) Final elongation | 72 | 300 | |

### RT-qPCR for Human and Murine AP4B1 Expression Analysis.

RT-qPCR was carried out using 2 μl total RNA diluted to a concentration of 10 ng/μl in nuclease free water, 5 μl 2× QuantiFast SYBR Green RT-PCR Master Mix (Qiagen®), hAP4B1 (Forward: 5′-CTGGTGAACGATGAGAATGT-3′ (SEQ ID NO 13); Reverse: 5′-GACCCAGCAACTCTGTTAAA-3′ (SEQ ID NO 14)), mAp4b1 (Forward: 5′-CTGTGCTAGGCTCCCACATC-3′(SEQ ID NO 15); Reverse: 5′-TGGCACTGGCCTTTACCATT-3′(SEQ ID NO 16)) and 18S (forward: 5′ GTAACCCGTTGAACCCCAT 3′ (SEQ ID NO 17); reverse: 5′ CCATCCAATCGGTAGTAGCG 3′(SEQ ID NO 18)) primers (all 1 μM concentration), 0.1 μl QuantiFast RT mix and H₂O to a final volume of 10 μl. Following an initial reverse transcription step at 50° C. for 10 min and a 5 min denaturation step at 95° C., cDNA was amplified by 39 cycles of 95° C. for 10 sec followed by a combined annealing/extension step at 60° C. for 10 sec. This was followed by one cycle at 65° C. for 31 sec, before subsequent melt curve analysis. All RT-qPCR was performed on a Bio-Rad C1000 Touch™ Thermal Cycler. Bio-Rad CFX Manager software was used to analyse signal intensity and relative gene expression values were determined using the ΔΔCt method, with 18S rRNA used as a reference gene.

### Open Field

Open field analysis was performed on mice at ages 6, 9 and 12 months. The protocol followed that performed by Herranz-Martin and colleagues⁸. Mice were placed in a translucent box with dimensions 60 cm×40 cm×25 cm. The underside of the box was marked with permanent ink outlining a 5×3 grid of squares. Activity was measured as the number of grid lines crossed by each mouse over a 10 minute period. For a crossing to be recorded, all four paws of the animal were required to cross the grid line. The assessment was carried out in minimal lighting conditions and the apparatus was cleaned with 70% ethanol between each animal. One run was recorded for each animal at each timepoint.

### Rotarod

Ugo Basile 7650 accelerating rotarod (set to accelerate from 3-37 rpm over 300 seconds) was used to measure motor function. Rotarod training was performed over 3 consecutive days, with two trials per day. Subsequently, this test was performed at bi-weekly intervals (characterisation study) or monthly (Proof-of-concept study) in the late morning. For each evaluation, the mice were tested twice, with a minimum rest period of 5 minutes between runs. The best performance, measured as latency to fall in seconds, was used for analysis. The minimum threshold for recording rotarod activity was 3 seconds.

### Gait Analysis

The CatWalk™ gait analysis system version 7.1 was used to assess gait parameters in Ap4b1-KO and WT mice. Mice were tested at 3, 6, 9 and 12 months of age. Mice were placed on the apparatus in complete darkness and their gait patterns recorded. Six unforced runs were recorded for each mouse and three selected for analysis. The runs to be analysed were selected based on the absence of behavioural anomalies—such as sniffing, exploration and rearing—and where mouse locomotion was consistent and without noticeable accelerations, decelerations or deviations from a straight line. Processing of gait data was performed with the Noldus software. Limbs were assigned manually, and gait parameters were calculated automatically. Parameter values were transferred to GraphPad Prism for statistical analysis.

### Antibodies

Primary antibodies used in this study were mouse anti-α-tubulin (1:5000; Sigma), mouse anti-GAPDH (1:10,000; Millipore), rabbit anti-V5 (1:1000; Abcam), rabbit anti-β4 (in-house non-commercial antibody provided by J. Hirst) (1:400), rabbit anti-ATG9A (1: 1000; Abcam), sheep anti-TGN46 (Bio-Rad), anti-MAP2.

### Protein Extraction and Western Blotting for Protein Expression Analysis.

Tissue was harvested from mice under terminal anaesthesia and snap frozen in liquid nitrogen. Tissue was homogenised using a dounce homogeniser in ice-cold RIPA buffer (50 mM Tris-HCL pH 7.4; 1% v/v NP-40; 0.5% w/v sodium deoxycholate; 0.1% v/v SDS; 150 mM NaCl; 2 mM EDTA) containing 1× protease inhibitor cocktail (Sigma-Aldrich). Lysate protein concentrations were determined using the BCA assay (Thermo Scientific Pierce™). 40 μg of protein lysate was denatured by heating to 100° C. for 5 minutes in the presence of 4× loading buffer (10 ml buffer contained: 240 mM Tris-HCL pH 6.8; 8% w/v SDS; 40% glycerol; 0.01% bromophenol blue; 10% β-mercaptoethanol). Lysates that were intended to be used for quantification of ATG9A protein levels were heated to 50° C., as boiling leads to aggregation of ATG9A and loss of signal. Lysates were then loaded onto 4-20% gradient mini-PROTEAN® TGX™ precast polyacrylamide gels (Bio-Rad). Gels were run at 180V in running buffer (25 mM Tris, 192 mM glycine, 0.1% SDS, pH 8.3) for approximately 50 minutes or until the dye front reached the bottom of the gel. Separated proteins were transferred by electrophoresis to an Immobilon-P PVDF membrane (Millipore) that had been pre-soaked in methanol. Protein transfer was carried out at 250 mA for 1.5 hours or 40 mA overnight in transfer buffer (25 mM Tris, 192 mM glycine, 5% v/v methanol). Membranes were blocked for 1 hour in 5% milk/TBS-T. Primary antibodies were diluted in 5% milk/TBS-T or 5% BSA/TBS-T and incubated with the membrane overnight at 4° C. After primary antibody incubation, the membrane was washed for 3×15 min each in TBS-T buffer. Secondary antibodies anti-mouse HRP (1:3000) and anti-rabbit HRP (1:3000) were diluted in 5% milk/TBS-T and incubated with the membrane for 2 hours at room temperature. After secondary antibody incubation, the membrane was washed for 3×15 min each in TBS-T buffer followed by a final 15 min wash in PBS. Protein bands were visualised using ECL Prime Western Blotting Detection Reagent (Amersham) and the G-Box imaging system (Syngene). Densitometric analysis of protein bands was carried out using Image J software.

Western blotting was generated using the following protocol: Cell lysates were extracted as above. 40 μg protein was loaded per lane on 10-well 4-12% Bis-Tris precast gel. The gel was run in 2-(N-morpholino) ethanesulfonic acid (MES) buffer and wet transferred to a nitrocellulose membrane (100 mA constant amps overnight). The membrane was blocked in 5% milk/TBS-T for 1 hour. Primary antibodies were added for 2 hours at room temperature (anti-AP4B1 1:400 in 5% BSA), followed by 4×15-minute washes in PBS-T. Secondary antibodies were added for 30 mins at room temperature in 5% milk-TBS-T. The membrane was washed 5×5 minutes in PBS-T followed by a 30-minute-long wash in PBS. The membrane was developed with ECL Prime Western Blotting Detection Reagent (Amersham).

### Cell Culture

Human Embryonic Kidney (HEK) 293T cells, HeLa-M/HeLa-AP4B1^{-/-} cells (a gift from Dr J. Hirst) and human fibroblast cell lines were cultured at 37° C., 5% CO₂ in growth media consisting of Dulbecco's Modified Eagle's Medium (DMEM, Sigma) supplemented with 10% v/v Fetal Bovine Serum (FBS, Sigma, MI, US) and 1% v/v penicillin (100 U/ml) and streptomycin (100 U/ml) (Lonza, Basel, Switzerland).

For primary cortical neuron culture, E18 non-transgenic rat embryos and E16 mouse embryos were harvested from wild type and C57BL/6J-Ap4b1^{em5Lutzy}/J pregnant mice, essentially as described by (Krichevsky et al., 2001). Very briefly, the cortices were dissected and digested in 0.25% trypsin in HBSS without calcium or magnesium (GIBCO) at 37° C. for 15 minutes and dissociated manually in triturating medium by using three fire-burnt Pasteur pipettes with successively smaller openings. Dissociated cortical neurons were then plated on poly-D-lysine (SIGMA) coated plates and maintained in Neurobasal medium (Life Technologies) supplemented with 2% B27 (Life Technologies), 0.5 mM GlutaMax (Life Technologies) and 100 U/ml of penicillin and 100 μg/ml streptomycin (Lonza).

AP4B1 knockout HeLa cells (HeLa-AP4B1^{-/-}) were provided by Dr J. Hirst, the generation of which is described in⁹.

The AP4B1 deficient human fibroblasts from SPG47 patients, heterozygous family members, and age-matched homozygous wild type controls were gifted by Dr Henry Houlden and Dr Ivy Pin-Fang Chen.

### Plasmid and Viral Construct Production for Off-Target Effects Analysis

AAV2-ITR transgene transfer plasmids—created as described above—were amplified in NEB Stable E. coli cells (New England Biolabs) and purified using Qiagen Plasmid Plus kits. Adenoviral helper genes (pHelper) and Rep-Cap genes (pAAV2/9) were supplied in trans and were obtained commercially through Plasmid Factory. Pseudotyped AAV9 viral vector was produced in-house following the protocol described in⁶.

### Validation of Viral Vectors In Vitro

HeLa-M and HeLa-AP4B1^{-/-} cells were transduced 24 hours after plating with viral vector mixed with normal growth media. Cells were incubated with virus for 3 days before being harvested for analysis.

Fibroblasts were transduced with a multiplicity of infection (MOI) of 20 lentiviral particles per cell. Cells were harvested 72 hours post-transduction.

Primary non-transgenic rat cortical neurons and murine cortical neurons harvested from wild type and C57BL/6J-Ap4b1^{em5Lutzy}/J mice were transduced with 300,000 vector genomes of viral vector 3 days after seeding. Cells were harvested 10 days post-transduction for ICC and Western blot/qPCR analysis of AP4B1A/5-AP4B1 and ATG9A expression.

### Vector Maps

TABLE 2
hSYN1-hAP4B1 (SEQ ID NO 19)
A. Vector summary

| | |
|---|---|
| Vector ID | VB210126-1113mgh |
| Vector Name | pAAV[Exp]-Kan-SYN1>hAP4B1[NM_001253852.3] |
| Vector Size | 6433 bp |
| Vector Type | Mammalian Gene Expression AAV Vector |
| Plasmid Copy Number | High |
| Antibiotic Resistance | Ampicillin |
| Cloning Host | VB UltraStable (or alternative strain) |

B. Vector components

| | | Size | | | |
|---|---|---|---|---|---|
| Name | Position | (bp) | Type | Description | Application notes |
| AAV2 ITR | 1-141 | 141 | misc_feature | None | None |
| SYN1 | 169-637 | 469 | misc_feature | None | full_name = |
| | | | | | Human synapsin I |
| | | | | | promoter |
| Kozak | 662-667 | 6 | misc_feature | None | full_name = Kozak |
| hAP4B1[NM_001253852.3] | 668-2887 | 2220 | misc_feature | None | None |
| BGH pA | 3530-3737 | 208 | misc_feature | None | full_name = Bovine |
| | | | | | growth hormone |
| | | | | | polyadenylation |
| 3′ ITR | 3745-3885 | 141 | ITR | AAV 3′ | note = Unknown |
| | | | | inverted | feature type: ITR |
| | | | | terminal | color: #0c5d90; |
| | | | | repeat | direction: RIGHT |
| f1 Origin | 3977-4283 | 307 | misc_feature | None | None |
| Kan | 4778-5587 | 810 | misc_feature | None | None |
| pUC ori | 5784-6372 | 589 | misc_feature | None | full_name = pUC |
| | | | | | origin of |
| | | | | | replication |

TABLE 3
CBh-hAP4B1 (SEQ ID NO 20)
A. Vector summary

| | |
|---|---|
| Vector ID | VB210118-1233uup |
| Vector Name | pAAV[Exp]-Kan-CBh>hAP4B1[NM_001253852.3] |
| Vector Size | 6174 bp |
| Vector Type | Mammalian Gene Expression AAV Vector |
| Plasmid Copy Number | High |
| Antibiotic Resistance | Ampicillin |
| Cloning Host | VB UltraStable (or alternative strain) |

B. Vector components

| | | Size | | | |
|---|---|---|---|---|---|
| Name | Position | (bp) | Type | Description | Application notes |
| AAV2 ITR | 1-141 | 141 | misc_feature | None | None |
| CBh | 169-966 | 798 | misc_feature | None | note = CBh |
| Kozak | 991-996 | 6 | misc_feature | None | note = Kozak |
| hAP4B1[NM_001253852.3] | 997-3216 | 2220 | misc_feature | None | note = |
| | | | | | hAP4B1[NM_001253852.3] |
| BGH pA | 3271-3478 | 208 | misc_feature | None | full_name = Bovine |
| | | | | | growth hormone |
| | | | | | polyadenylation |
| 3′ ITR | 3486-3626 | 141 | ITR | AAV 3′ | None |
| | | | | inverted | |
| | | | | terminal | |
| | | | | repeat | |
| f1 Origin | 3718-4024 | 307 | misc_feature | None | None |
| Kan | 4519-5328 | 810 | misc_feature | None | None |
| pUC ori | 5525-6113 | 589 | misc_feature | None | full_name = pUC |
| | | | | | origin of |
| | | | | | replication |

TABLE 4
SYN 1hAP4S1-3′UTR (SEQ ID No 21)
A Vector summary

| | |
|---|---|
| Vector ID | VB210304-1223zrw |
| Vector Name | pscAAV[Exp]-Kan-SYN1>hAP4S1[NM_001128126.3]:3′UTR_790bp |
| Vector Size | 5255 bp |
| Viral Genome Size | 2297 bp |
| Vector Type | Mammalian Gene Expression scAAV Vector |
| Plasmid Copy Number | High |
| Antibiotic Resistance | Kanamycin |
| Cloning Host | VB UltraStable (or alternative strain) |

B.Vector components

| | | Size | | | |
|---|---|---|---|---|---|
| Name | Position | (bp) | Type | Description | Application notes |
| 5′ ITR | 272-414 | 143 | misc_feature | None | None |
| SYN1 | 460-928 | 469 | misc_feature | None | note = SYN1 |
| Kozak | 953-958 | 6 | misc_feature | | note = Kozak |
| hAP4S1[NM_001128126.3] | 959-1393 | 435 | misc_feature | None | note = |
| | | | | | hAP4S1[NM_001128126.3] |
| 3′UTR_790bp | 1394-2183 | 790 | misc_feature | None | note = 3′UTR_790bp |
| SV40 poly(A) signal | 2237-2358 | 122 | polyA_signal | None | None |
| 3′ ITR-Deltatrs | 2456-2568 | 113 | misc_feature | None | None |
| Sall | 2588-2593 | 6 | misc_feature | None | None |
| KanR | 3480-4289 | 810 | misc_feature | None | None |
| ori | 4486-5074 | 589 | rep_origin | None | full_name = pUC |
| | | | | | origin of |
| | | | | | replication |

TABLE 5
MeP229hAP4S1 (SEQ ID NO 22)
A.Vector summary

| | |
|---|---|
| Vector ID | VB210208-1245mxd |
| Vector Name | pscAAV[Exp]-Kan-{MeP229}>hAP4S1[NM_001128126.3] |
| Vector Size | 3909 bp |
| Vector Type | Mammalian Gene Expression scAAV Vector |
| Plasmid Copy Number | High |
| Antibiotic Resistance | Kanamycin |
| Cloning Host | VB UltraStable (or alternative strain) |

B.Vector components

| | | Size | | | |
|---|---|---|---|---|---|
| Name | Position | (bp) | Type | Description | Application notes |
| 5′ ITR | 1-143 | 143 | ITR | AAV 5′ inverted | note = Unknown |
| | | | | terminal repeat | feature type: ITR |
| | | | | | color: #c54b7c; |
| | | | | | direction: RIGHT |
| {MeP229} | 189-413 | 225 | Promoter | None | note = Unknown |
| | | | | | feature |
| | | | | | type: promoter |
| | | | | | color: #d05c0a; |
| | | | | | direction: RIGHT |
| Kozak | 438-443 | 6 | Miscellaneous | Kozak | full_name = Kozak |
| | | | | translation | note = Unknown |
| | | | | initiation | feature |
| | | | | sequence | type: Miscellaneous |
| | | | | | color: #fd3434; |
| | | | | | direction: RIGHT |
| hAP4S1[NM_001128126.3] | 444-878 | 435 | misc_feature | None | note = |
| | | | | | hAP4S1[NM_001128126.3] |
| SV40 late pA | 923-1144 | 222 | PolyA_signal | Simian virus 40 | note = Unknown |
| | | | | late | feature |
| | | | | polyadenylation | type: PolyA_signal |
| | | | | signal | color: #46c6ef; |
| | | | | | direction: RIGHT |
| 3′ ITR-Del trs | 1151-1263 | 113 | ITR | AAV3′ITRwith a | note = Unknown |
| | | | | depleted | feature type: ITR |
| | | | | terminal | color: #eda481; |
| | | | | resolution site | direction: RIGHT |
| KanR | 1889-2698 | 810 | misc_feature | None | note = KanR |
| pUC ori | 2869-3457 | 589 | Rep_origin | pUC origin of | full_name = pUC |
| | | | | replication | origin of |
| | | | | | replication |
| | | | | | note = Unk |

TABLE 6
MeP229hAP4E1 (SEQ ID NO 23)
A Vector summary

| | |
|---|---|
| Vector ID | VB210208-1233mtr |
| Vector Name | pAAV[Exp]-Kan-{MeP229}>hAP4E1[NM_007347.5] |
| Vector Size | 6795 bp |
| Viral Genome Size | 4247 bp |
| Vector Type | Mammalian Gene Expression AAV Vector |
| Plasmid Copy Number | High |
| Antibiotic Resistance | Kanamycin |
| Cloning Host | VB UltraStable (or alternative strain) |

B.Vector components

| | | Size | | | |
|---|---|---|---|---|---|
| Name | Position | (bp) | Type | Description | Application notes |
| 5′ ITR | 1-141 | 141 | ITR | AAV 5′ inverted | note = Unknown |
| | | | | terminal repeat | feature |
| | | | | (functional | type: ITR |
| | | | | equivalent of | color: #a949ca; |
| | | | | wild-type 5′ | direction: RIGHT |
| | | | | ITR) | |
| MeP229 | 169-393 | 225 | misc_feature | None | note = MeP229 |
| Kozak | 418-423 | 6 | misc_feature | None | full_name = Kozak |
| hAP4E1[NM_007347.5] | 424-3837 | 3414 | misc_feature | | note = |
| | | | | | hAP4E1[NM_007347.5] |
| BGH pA | 3892-4099 | 208 | misc_feature | None | full_name = Bovine |
| | | | | | growth hormone |
| | | | | | polyadenylation |
| 3′ ITR | complement | 141 | ITR | AAV 3′ | note = Unknown |
| | (4107-4247) | | | inverted | feature |
| | | | | terminal | type: ITR |
| | | | | repeat | color: #db6901; |
| | | | | | direction: LEFT |
| KanR | 5140-5949 | 810 | misc_feature | None | None |
| ori | 6146-6734 | 589 | misc_feature | None | full_name = pUC |
| | | | | | origin of |
| | | | | | replication |

TABLE 7
SYN1hAP4E1 (SEQ ID NO 24)
A. Vector summary

| | |
|---|---|
| Vector ID | VB210212-1131ddf |
| Vector Name | pAAV[Exp]-Kan-SYN1>hAP4E1[NM_007347.5] |
| Vector Size | 7039 bp |
| Vector Type | Mammalian Gene Expression AAV Vector |
| Plasmid Copy Number | High |
| Antibiotic Resistance | Kanamycin |
| Cloning Host | VB UltraStable (or alternative strain) |

B.Vector components

| | | Size | | | |
|---|---|---|---|---|---|
| Name | Position | (bp) | Type | Description | Application notes |
| nonstandard | 1-141 | 141 | ITR | [AAV 5′ | note = Unknown |
| type: ITR | | | | inverted | feature type: ITR |
| | | | | terminal | color: #7a00b5; |
| | | | | repeat | direction: RIGHT |
| | | | | (functional | |
| | | | | equivalent of | |
| | | | | wild-type 5′ | |
| | | | | ITR)] | |
| SYN1 | 169-637 | 469 | misc_feature | None | note = SYN1 |
| Kozak | 662-667 | 6 | misc_feature | None | full_name = Kozak |
| hAP4E1[NM_007347.5] | 668-4081 | 3414 | misc_feature | None | None |
| BGH pA | 4136-4343 | 208 | misc_feature | None | full_name = Bovine |
| | | | | | growth hormone |
| | | | | | polyadenylation |
| nonstandard | complement | 141 | ITR | [AAV 3′ | note = Unknown |
| type: ITR | (4351-4491) | | | inverted | feature |
| | | | | terminal | type: ITR |
| | | | | repeat] | color: #6d3ed8; |
| | | | | | direction: LEFT |
| KanR | 5384-6193 | 810 | misc_feature | None | None |
| ori | 6390-6978 | 589 | misc_feature | None | full_name = pUC |
| | | | | | origin of |
| | | | | | replication |

### EXAMPLE 1

The size of the human AP4B1 cDNA open reading frame (2,800 bp) means that a simple gene replacement option is technically feasible and amenable to typical viral delivery approaches such as using a single-stranded adeno-associated virus (AAV) which has an insertion limit of 4,000 bp. We designed AAV vectors to achieve the strongest level of transgene expression (FIG. 2A): 1) An expression cassette was developed involving the 0.8 kb CBh promoter and 130 bp SV40 polyA to drive expression of the human AP4B1. The CBh promoter has been reported to mediate efficient transgene expression in rodents and non-human primates; 2) A vector expressing an N-terminal V5 viral epitope-tagged human AP4B1 cDNA allowing in vitro and in vivo detection of AP4B1 restoration in the absence of suitable anti-AP4B1 antibodies; 3) A V5-tagged AP4B1 construct expressed from a lentiviral vector enabling in vitro validation of efficacy in cell types that are not efficiently transduced by AAV9 (e.g. fibroblasts). All constructs have been shown to efficiently express their viral cargos upon transfection or transduction in HeLa cells (FIG. 2B,C), primary rat cortical neurons (FIG. 2D), and human fibroblasts (FIG. 2E) as determined by western blotting, RT-qPCR and immunocytochemistry. The viral constructs efficiently restore expression of the AP4B1 protein in both a CRISPR generated AP4B1-knockout HeLa cell line and fibroblasts from SPG47 patients lacking endogenous AP4B1 (FIG. 2 B,C,E). Expression of V5-tagged AP4B1 in SPG47 patient fibroblasts also rescues both overexpression and mis localisation of ATG9A (FIG. 3).

### EXAMPLE 2

Intrathecal delivery of AAV9 viral vectors (AAV9-AP4B1, AAV9-V5-tagged-AP4B1 or AAV9-GFP) via the cisterna magna in wildtype C57BL6/J mice, resulted in widespread transduction of multiple tissues including the brain (FIG. 4A,B), a route of delivery known to lead to efficient gene transfer to CNS as described in our previous work. Long-term pilot safety studies in wild type mice treated with AAV9-AP4B1 show no apparent side effects on body weight, motor function or clinical observations up to the age of 6 months (FIG. 4C-E).

### EXAMPLE 3

In tandem with the development of the therapeutic viral vector, generation of CRISPR-based Ap4b1-knockout (EM5 Ap4b1−/−) mouse line was outsourced to the Jackson Laboratory then transferred to SITraN in Sheffield for full characterisation. Phenotypic characterisation of the line revealed that EM5 Ap4b1−/− lines show consistent progressive motor deficits as demonstrated by roratod, open field and CatWalk footprint testing (FIG. 5A-C) and clasping (FIG. 6). Preliminary MRI analysis revealed thin corpus callosum in of EM5 Ap4b1−/− when compared to wild type mice (FIG. 7A,B), a clinically relevant phenotype since the same pathological phenotype has been reported in SPG47 patients. This observation has been confirmed by H & E staining (FIG. 7C,D). Further analysis of MRI study is ongoing to assess other readouts such as the overall size of lateral ventricles, and alterations in white matter volume, phenotypes already reported in AP4E1−/− and human AP4 cases.

A mouse model of SPG47 was generated by Jackson labs (Bar Harbor, Me.): The C57BL/6J-Ap4b1^{em5Lutzy}/J model (Stock #031349) contains a mutant Ap4b1 gene with a 76 bp deletion within exon 1. The C57BLJ6J-Ap4b1^{em4Lutzy}/J model (Stock #031062) contains a 78 bp deletion+1 bp deletion in exon 1 of mouse Ap4b1 gene. Neither is known to be a human pathogenic mutation but are predicted to generate frameshift that result in early nonsense mutations. The strain was developed with CRISPR/Cas 9 technology and a mutagenic oligonucleotide. Plasmids encoding a signal guide RNA designed to introduce a 76 bp deletion within exon 1 of the Ap4b1 gene and the cas9 nuclease were introduced into the cytoplasm C57BL/6J-derived fertilized eggs with well recognized pronuclei. Correctly targeted embryos were transferred to pseudopregnant females. Correctly targeted pups were identified by sequencing and PCR and further bred to C57BL/6J to develop the colony. PCR genotyping allows identification of wildtype, heterozygotes and homozygous knockout mice.

Further characterization of the model was executed by Azzouz lab of University of Sheffield. RT-PCR and qRT-PCR showed very low levels of Ap4b1 mRNA in the mutant mice consistent with nonsense mediate decay. Western blots showed the absence of a cross reacting band of ˜85 kDa normally expressed at various levels in brain, muscle, spinal cord, liver and heart of wildtype mice.

The em5lutzy line has been examined in more detail. Weight gain in wildtype and Ap4b1−/− female mice is similar while weight gain in male Ap4b1−/− mice is slower than male wildtype mice with a significantly lower weight at >6 months. A number of behavioral assessments were conducted including gait analysis clasping, open field testing and rotarod performance (FIGS. 5 and 6). All of these showed a deficiency in the Ap4b1−/− mice. Gait analysis showed paw angle for hind limbs was abnormally wide for mutant mice compared to wildtype (FIG. 5D). In the clasping assay mutant mice had a higher percent clasping at all ages compared with wildtype (FIG. 6B). Open field activity was comparable in wildtype and mutant mice at 6 months of age but there was age-dependent decrease in wildtype mice that was absent in mutant mice (FIG. 5B). The rotarod showed the most consistent difference with latency to fall on rotarod is ˜15% lower at all ages tested form 50 to 120 days of age (FIG. 5A). Further characterization is underway by open field activity monitoring, catwalk, hind limb clasping, MRI, histopathology, ATG9A localization and protein levels. Motor deficiency is a key feature of the clinical presentation of SPG47/AP4B1 deficiency and therefore these mouse phenotypes are directly relevant to the human disease and appropriate markers for assessing potential therapeutics.

Further morphological and histopathological studies on these Ap4b1−/− knockout mice are ongoing. A published study on a knockout mouse for the Ap4e1 gene shows a thin corpus callosum and axonal swellings in various areas of the brain and spinal cord. Immunohistochemical analyses showed that the transmembrane autophagy-related protein 9A (ATG9A) is more concentrated in the trans-Golgi network (TGN) and depleted from the peripheral cytoplasm both in various neuronal types in Ap4e1 knockout mice. This leads to distal axonal swellings containing accumulated ER, defective autophagosomes, and shortening of the axons observable both in vitro and in vivo.

Proof of Concept. Treatment of SPG47 Mouse Model by Intra-Cisterna Magna AAV9

Three studies have been conducted to assess the impact of intra-cisterna magna AAV9-hAP4B1 in mouse C57BL/6J-Ap4b1^{em5Lutzy}/J model using juvenile of neonatal mice.

### Cisterna Magna Injection of p1 Mice.

Proof of concept was demonstrated in newborn Ap4b1−/− mice. By using neonates, it was assumed that there would be maximum benefit to relieve any developmental manifestations of disease. Experimental design (1) included 4 cohorts of homozygous knockout mice with either of two doses of the AAV9-HAP4B1 vector expressing human cDNA for AP4B1; empty vector expressing epitope tag (AAV9-V5) or untreated. Positive control was untreated wildtype mice.

Stocks of AAV9 viral vectors were produced by transfecting adherent human embryonic kidney HEK293T cells and purification of the vector using iodixanol gradient centrifugation. Briefly, HEK293T cells were transfected with packaging plasmids pHelper (Stratagene; Stockport, UK), pAAV2/9 (kindly provided by J. Wilson, University of Pennsylvania) and one of the transgene plasmids (e.g. AAV9-CBh-AP4B1) at 2:1:1 ratio, respectively, using polyethylenimine (1 mg/ml) in serum-free Dulbecco's modified Eagle's medium. At 3 days post-transfection, supernatant containing cell-released virus was harvested, treated with benzonase (10 unit/ml; Sigma, Poole, UK) for 2 hours at 37° C. and concentrated to equal to approximately 24 ml using Amicon Ultra-15 Centrifugal 100K Filters (Millipore, Watford, UK). Iodixanol gradient containing 15, 25, 40, and 54% iodixanol solution in phosphate-buffered saline (PBS)/1 mmol/1 MgCl2/2.5 mmol/l KCl and virus solution was loaded and centrifuged at 69,000 revolutions per minute for 90 minutes at 18° C. After ultracentifugation, the virus fractions were visualized on a 10% polyacrylamide gel, stained using SYPRO Ruby (Life Technologies, Paisley, UK) according to the manufacturer's guidelines. The highest purity fractions (identified by the presence of the three bands corresponding to VP1, VP2, and VP3) were pooled and concentrated further in the final formulation buffer consisting of PBS supplemented with an additional 35 mmol/l NaCl using Amicon Ultra-15 Centrifugal 100K filters. Viral titers were determined by quantitative PCR assays.

TABLE 1
Study design for P1 mouse efficacy experiment¹

| | | | N | |
|---|---|---|---|---|
| | | | (Male/ | Total |
| Cohort | Genotype | Treatment/dose | Female) | N |
| 1 | Ap41b1−/− | AAV9-hAP4B1 (5 × 10¹³ gc/kg) | 6/4 | 10 |
| 2 | Ap41b1−/− | AAV9-hAP4B1 (2 × 10¹³ gc/kg) | 8/1 | 9 |
| 3 | Ap41b1−/− | AAV9-V5 (5 × 10¹³ gc/kg) | 5/4 | 9 |
| 4 | Wildtype | Untreated | 9/8 | 17 |
| 5 | Ap41b1−/− | Untreated | 1/6 | 7 |

¹Assessments included general observation, weight and accelerating rotarod every 3 weeks, open field, clasping and catwalk testing at 3-month intervals.

There was no mortality observed for any mice in any of these cohorts. Weigh gain was assessed every 2 weeks (FIG. 9) with a clear pattern of slower weight gain by untreated Ap4b1−/− mice of either gender compared to wildtype controls. Neonatal injection of high dose AAV9-hAP4B1 restored male weight gain to levels in wildtype mice whereas it had no impact on weight gain in female mice.

Phenotype was assessed by the clasping assay in which mice with certain neurological defects (but not wildtype mice) clasp their limbs when suspended by the tail. The data (FIG. 10) clearly show an age-dependent increase with 83% of knockout mice showing clasping response compared to 7% wildtype controls at 9-month age (p<0.001; Chi square test). Treatment of knockout mice by a high dose of AAV9-hAP4B1 vector significantly mitigated clasping response at 9 months (p<0.01) although response did not achieve the level in wildtype mice (p<0.0001). By contrast, control vector AAV9-CBH-V5, had no impact on clasping response. Also, use of low dose AAV9-hAP4B1 showed no impact with age dependent clasping comparable to that of untreated Ap4b1−/− mice suggesting that the target dose is at least 5×10¹³ gc/kg.

Phenotype was also assessed by accelerating rotarod (FIG. 11) at 4-week intervals. As with weight gain, the data are more clearly understood when male and female mice are considered separately. For male mice there was clear improvement in performance of high dose AAV9-hAP4B1 treated knockouts up to a level comparable to that of wildtype mice of the same age. For female mice, the knockouts did not show a defect in rotarod performance and therefore there is no possibility of seeing any response to AAV-hAP4B1 delivery into cistern magna. We note that substantial differences in performance between males and female mice are known in both wildtype [12] and mouse models of neurological diseases including GNAO1 [13], ALS [14, 15] and Alzheimer's [16].

Performance was also assessed by overall activity in open field testing in a subset of mice. By this measure there was a clear difference between wildtype and knockout mice but no impact of treatment on activity. A similar pattern was seen in both male and female cohorts.

Status of the AP4 complex was assessed by measuring the AP4E1 levels (FIG. 12). An important observation is the depletion of AP4E1 unit of the AP4 complex in AP4b1^{-/-} when comparison to WT mice. The same observation has been reported in cell model systems of

SPG47 including human cells isolated from AP4 patients. AAV9-AP4B1 gene transfer increase AP4E1 levels in the AP4B1−/− mouse spinal cord (FIG. 12A) and Heart (FIG. 12B) suggesting restoration of AP4 complex (FIG. 13).

CM delivery in P1 wild type mice: Pilot Safety Study

This aim of this pilot study was to assess the biodistribution, stability of viral-mediated transgene expression and potential adverse effects of AAV9-hAP4B1 in wildtype mice (FIG. 4). One viral vector will be used, expressing a full-length copy of the human AP4B1 gene containing an N-terminal V5 viral epitope tag. The viral vectors were delivered directly into the cisterna magna of P1/2 pups, using stereotaxic apparatus containing a 33-gauge Hamilton syringe with automated perfusion pump. Five p of solution was administered at 1 uL per minute. Biodistribution of the virus, expression, body weight and motor function (rotarod) were assessed at 4 weeks and 6 months post injection.

TABLE 8
Sequence summary

| SEQ | |
|---|---|
| ID NO | Description |
| 1 | Homo sapiens adaptor related protein complex 4 subunit beta 1 |
| | (AP4B1)-DNA |
| 2 | AP4B1-Amino acid sequence |
| 3 | Homo sapiens adaptor related protein complex 4 subunit epsilon |
| | 1 (AP4E1)-DNA |
| 4 | AP4E1-Amino acid sequence |
| 5 | Homo sapiens adaptor related protein complex 4 subunit sigma 1 |
| | (AP4M1)-DNA |
| 6 | AP4M1-Amino acid sequence |
| 7 | Homo sapiens adaptor related protein complex 4 subunit mu 1 |
| | (AP4S1)-DNA |
| 8 | AP4S1-Amino acid sequence |
| 9 | CRISPR-sequence |
| 10-18 | Primer sequences |
| 19 | hSYN1-hAP4B1 |
| 20 | CBh-hAP4B1 |
| 21 | SYN 1hAP4S1-3'UTR |
| 22 | MeP229hAP4S1 |
| 23 | MeP229hAP4E1 |
| 24 | SYN1hAP4E1 |
| 25 | Stuffer sequence 1 |
| 26 | Stuffer sequence 2 |
| 27 | AP4 promoter |
| 28 | AP4 promoter forward primer |
| 29 | AP4 promoter reverse primer |
| 30 | Mininmal promoter sequence |

### REFERENCES

1 Harvard SPG47 registry, managed by Dr Darius Ebrahimi-Fakhari, accessed October 2020.
2 Bauer, P. et al. Mutation in the AP4B1 Gene Cause Hereditary Spastic Paraplegia Type47 (SPG47). Neurogenetics 13, 73-76, doi:10.1007/s10048-012-0314-0. (2012).
3. Laughlin, C. A., Tratschin, J. D., Coon, H. & Carter, B. J. Cloning of infectious adeno-associated virus genomes in bacterial plasmids. Gene 23, 65-73 (1983).
4. Gray, S. J. et al. Optimizing Promoters for Recombinant Adeno-Associated Virus-Mediated Gene Expression in the Peripheral and Central Nervous System Using Self-Complementary Vectors. Hum. Gene Ther. 22, 1143-1153 (2011).
5. Davies, A. K. et al. AP-4 vesicles contribute to spatial control of autophagy via RUSC-dependent peripheral delivery of ATG9A. Nat. Commun. 9, (2018).
6. Lukashchuk, V., Lewis, K. E., Coldicott, I., Grierson, A. J. & Azzouz, M. AAV9-mediated central nervous system-targeted gene delivery via cisterna magna route in mice. Mol. Ther.—Methods Clin. Dev. 3, 15055 (2016).
7. Kim, S., Yu, N. K. & Kaang, B. K. CTCF as a multifunctional protein in genome regulation and gene expression. Exp. Mol. Med. 47, e166 (2015).
8. Herranz-Martin, S. et al. Viral delivery of C9orf72 hexanucleotide repeat expansions in mice leads to repeat-length-dependent neuropathology and behavioural deficits. Dis. Model. Mech. 10, 859-868 (2017).
9. Frazier, M. N. et al. Molecular basis for the interaction between Adaptor Protein Complex 4 (AP4) β4 and its accessory protein, tepsin. Traffic 17, 400-415 (2016).
10. Behne R, Teinert J, Wimmer M, D'Amore A, Davies A K, Scarrott J M, Eberhardt K, Brechmann B, Chen I P, Buttermore E D, Barrett L, Dwyer S, Chen T, Hirst J, Wiesener A, Segal D, Martinuzzi A, Duarte S T, Bennett J T, Bourinaris T, Houlden H, Roubertie A, Santorelli F M, Robinson M, Azzouz M, Lipton J O, Borner GHH, Sahin M, Ebrahimi-Fakhari D. Adaptor protein complex 4 deficiency: a paradigm of childhood-onset hereditary spastic paraplegia caused by defective protein trafficking. Hum Mol Genet. 2020 Jan. 15; 29(2):320-334. doi: 10.1093/hmg/ddz310. PMID: 31915823; PMCID: PMC7001721.
11. Ebrahimi-Fakhari D, Teinert J, Behne R, Wimmer M, D'Amore A, Eberhardt K, Brechmann B, Ziegler M, Jensen D M, Nagabhyrava P, Geisel G, Carmody E, Shamshad U, Dies K A, Yuskaitis C J, Salussolia C L, Ebrahimi-Fakhari D, Pearson T S, Saffari A, Ziegler A, Kölker S, Volkmann J, Wiesener A, Bearden D R, Lakhani S, Segal D, Udwadia-Hegde A, Martinuzzi A, Hirst J, Perlman S, Takiyama Y, Xiromerisiou G, Vill K, Walker W O, Shukla A, Dubey Gupta R, Dahl N, Aksoy A, Verhelst H, Delgado M R, Kremlikova Pourova R, Sadek A A, Elkhateeb N M, Blumkin L, Brea-Fernandez A J, Dacruz-Alvarez D, Smol T, Ghoumid J, Miguel D, Heine C, Schlump J U, Langen H, Baets J, Bulk S, Darvish H, Bakhtiari S, Kruer M C, Lim-Melia E, Aydinli N, Alanay Y, El-Rashidy O, Nampoothiri S, Patel C, Beetz C, Bauer P, Yoon G, Guillot M, Miller S P, Bourinaris T, Houlden H, Robelin L, Anheim M, Alamri A S, Mahmoud AAH, Inaloo S, Habibzadeh P, Faghihi M A, Jansen A C, Brock S, Roubertie A, Darras B T, Agrawal P B, Santorelli F M, Gleeson J, Zaki M S, Sheikh S I, Bennett J T, Sahin M. Defining the clinical, molecular and imaging spectrum of adaptor protein complex 4-associated hereditary spastic paraplegia. Brain. 2020 Oct. 1; 143(10):2929-2944. doi: 10.1093/brain/awz307. PMID: 32979048; PMCID: PMC7780481.
12. Orsini, C.A. and B. Setlow, Sex differences in animal models of decision making. J Neurosci Res, 2017. 95(1-2): p. 260-269.
13. Feng, H., et al., Mouse models of GNAO1-associated movement disorder: Allele- and sex-specific differences in phenotypes. PLoS One, 2019. 14(1): p. e0211066.
14. McCombe, P. A. and R. D. Henderson, Effects of gender in amyotrophic lateral sclerosis. Gend Med, 2010. 7(6): p. 557-70.
15. Watkins, J., et al., Female sex mitigates motor and behavioural phenotypes in TDP-43(Q331K) knock-in mice. Sci Rep, 2020. 10(1): p. 19220.
16. Sala Frigerio, C., et al., The Major Risk Factors for Alzheimer's Disease: Age, Sex, and Genes Modulate the Microglia Response to Aβ Plaques. Cell Rep, 2019. 27(4): p. 1293-1306.e6.

1. An isolated nucleic acid molecule comprising: a transcription cassette comprising a promoter adapted for expression in a mammalian neurone said cassette further comprising a nucleic acid molecule comprising a nucleotide sequence that encodes at least one protein of the AP-4 complex.

2. The isolated nucleic acid molecule according to claim 1, comprising a nucleic acid molecule comprising a nucleotide sequence selected from the group consisting of:
i) a nucleotide sequence, or polymorphic sequence variant, as set forth in SEQ ID NO:1 (AP4B1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 1 (AP4B1) wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 2 (AP4B1);
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

3. The isolated nucleic acid molecule according to claim 1, comprising a nucleic acid molecule comprising a nucleotide sequence selected from the group consisting of:
i) a nucleotide sequence, or polymorphic sequence variant, as set forth in SEQ ID NO:3 (AP4E1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 3 (AP4E1) wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 4 (AP4E1);
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

4. The isolated nucleic acid molecule according to claim 1, comprising a nucleic acid molecule comprising a nucleotide sequence selected from the group consisting of:
i) a nucleotide sequence, or polymorphic sequence variant, as set forth in SEQ ID NO: 5 (AP4M1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 5 (AP4M1) wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 6 (AP4M1);
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

5. The isolated nucleic acid molecule according to claim 1, comprising a nucleic acid molecule comprising a nucleotide sequence selected from the group consisting of:
i) a nucleotide sequence, or polymorphic sequence variant, as set forth in SEQ ID NO: 7 (AP4S1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 7 (AP4S1) wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 8 (AP4S1);
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

6. The isolated nucleic acid molecule according to any one of claims 1 to 5, wherein said cassette is adapted for expression in a motor neurone.

7. The isolated nucleic acid molecule according to claim 6 wherein said promoter is selected from the group consisting of: chicken beta actin (CBA) promoter, chicken beta actin hybrid (CBh) promoter, CAG promoter, neuronal and glial specific promoters including synapsin 1, Hb9, MeP229 and GFAP promoter sequences, as well as AP-4 subunit specific promoter regions including AP4B1, AP4E1, AP4M1 and AP4S1.

8. The isolated nucleic acid molecule according to claim 7 wherein said promoter sequence comprises a nucleic acid molecule comprising a nucleotide sequence as set forth in SEQ ID NO: 27, or a nucleotide sequence that is a polymorphic sequence variant of SEQ ID NO: 27.

9. An expression vector comprising an isolated nucleic acid molecule according to any one of claims 1 to 8.

10. The expression vector according to claim 9, wherein said expression vector is a viral based expression vector.

11. The expression vector according to claim 10 wherein said viral based vector is AAV9.

12. The expression vector according to claim 10 wherein said viral based vector is a lentiviral vector.

13. The expression vector according to claim 9 wherein said viral based vector comprises the nucleotide sequence set forth in SEQ ID NO: 19 (AP4B1).

14. The expression vector according to claim 9 wherein said viral based vector comprises the nucleotide sequence set forth in SEQ ID NO: 20 (AP4B1).

15. The expression vector according to claim 9 wherein said viral based vector comprises the nucleotide sequence set forth in SEQ ID NO: 21 (AP4S1).

16. The expression vector according to claim 9 wherein said viral based vector comprises the nucleotide sequence set forth in SEQ ID NO: 22 (AP4S1).

17. The expression vector according to claim 9 wherein said viral based vector comprises the nucleotide sequence set forth in SEQ ID NO: 23 (AP4E1).

18. The expression vector according to claim 9 wherein said viral based vector comprises the nucleotide sequence set forth in SEQ ID NO: 24 (AP4E1).

19. The expression vector according to any one of claims 9 to 12—wherein said viral based vector comprises further the nucleotide sequence set forth in SEQ ID NO: 25 or 26.

20. A pharmaceutical composition comprising an expression vector according to any one of claims 9 to 19 and an excipient or carrier.

21. An expression vector according to any one of claims 9 to 19 for use as a medicament.

22. An expression vector according to any one of claims 9 to 19 for use in the treatment of AP-4-Hereditary Spastic Paraplegia (AP-4-HSP).

23. The expression vector according to the use of claim 22 wherein the AP-4-HSP is SPG47, SPG50, SPG51 or SPG52.

24. A cell transfected with an expression vector according to any one of claims 9 to 19.

25. The cell according to claim 24 wherein said cell is a neurone.

26. The cell according to claim 25 wherein said cell is a motor neurone.

27. A method to treat or prevent AP-4 Hereditary Spastic Paraplegias (HSP) comprising administering a therapeutically effective amount of an expression vector according to any one of claims 9 to 19 to prevent and/or treat AP-4-HSP.

28. The method according to claim 27 wherein said AP-4-HSP is SPG47, SPG50, SPG51 or SPG52.

29. A diagnostic method to genotype a subject to determine whether the subject has a mutation in one or more AP-4 gene sequences comprising the steps:
i) obtaining a biological sample from a subject to be tested and extracting nucleic acid from said biological sample;
ii) sequencing the nucleic acid to obtain a nucleotide sequence of AP4B1, AP4E1, AP4M1 and AP4S1 in said subject;
iii) comparing the obtained genomic sequence with a normal matched control nucleotide sequence to identify nucleotide sequence differences; and
iv) determining whether the test sample is modified in an AP-4 gene sequence and whether said modification is associated with AP-4-HSP.

30. The diagnostic method according to claim 29, wherein said method further comprises the administration of at least one expression vector according to any one of claims 9 to 19 to prevent or treat AP-4-HSP.

31. The diagnostic method according to claim 29 or 30, wherein said AP-4-HSP is selected from the group consisting of: SPG47, SPG50, SPG51 and SPG52.

32. The diagnostic method according to any one of claims 29 to 31, wherein said genomic sequence comprises SEQ ID NO: 1, SEQ ID NO: 3, SEQ ID NO: 5 or SEQ ID NO: 7, or a polymorphic sequence variant thereof.

33. A transcription cassette comprising:
a first nucleic acid molecule comprising a nucleotide sequence as set forth in SEQ ID NO: 27, or a nucleotide sequence that is a polymorphic sequence variant of SEQ ID NO: 27, wherein said nucleic acid molecule is a transcription promoter and operably linked to a second nucleic acid molecule comprising a nucleotide sequence encoding a polypeptide wherein the first nucleic acid molecule regulates transcription of the second nucleic acid molecule.

34. The transcription cassette according to claim 33 wherein said first nucleic acid molecule comprises or consists of the nucleotide sequence set forth in SEQ ID NO: 27.

35. The transcription cassette according to claim 33 or 34 wherein said second nucleic acid molecule comprises a nucleotide sequence that encodes at least one polypeptide of the AP-4 complex.

36. The transcription cassette according to claim 35 wherein said second nucleic acid molecule comprises or consists of a nucleotide sequence as represented in SEQ ID NO: 1, or polymorphic sequence variant thereof.

37. The transcription cassette according to claim 35 wherein said nucleic acid molecule comprises or consists of a nucleotide sequence as represented in SEQ ID NO: 3, or polymorphic sequence variant thereof.

38. The transcription cassette according to claim 35 wherein said nucleic acid molecule comprises or consists of a nucleotide sequence as represented in SEQ ID NO: 5, or polymorphic sequence variant thereof.

39. The transcription cassette according to claim 35 wherein said nucleic acid molecule comprises or consists of a nucleotide sequence as represented in SEQ ID NO: 7, or polymorphic sequence variant thereof.

40. An expression vector comprising a transcription cassette according to any one of claims 33 to 39.

41. The expression vector according to claim 40, wherein said expression vector is a viral based expression vector.

42. The expression vector according to claim 41 wherein said viral based vector is AAV9.

43. The expression vector according to claim 41 wherein said viral based vector is a lentiviral vector.


SEQUENCE LISTING (30 sequences)

SEQ ID NO: 1 - DNA, 2220 bp, Homo sapiens
atgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataa

SEQ ID NO: 2 - protein, 739 aa, Homo sapiens
MPYLGSEDVVKELKKALCNPHIQADRLRYRNVIQRVIRYMTQGLDMSGVFMEMVKASATVDIVQKKLVYLYMCTYAPLKPDLALLAINTLCKDCSDPNPMVRGLALRSMCSLRMPGVQEYIQQPILNGLRDKASYVRRVAVLGCAKMHNLHGDSEVDGALVNELYSLLRDQDPIVVVNCLRSLEEILKQEGGVVINKPIAHHLLNRMSKLDQWGQAEVLNFLLRYQPRSEEELFDILNLLDSFLKSSSPGVVMGATKLFLILAKMFPHVQTDVLVRVKGPLLAACSSESRELCFVALCHVRQILHSLPGHFSSHYKKFFCSYSEPHYIKLQKVEVLCELVNDENVQQVLEELRGYCTDVSADFAQAAIFAIGGIARTYTDQCVQILTELLGLRQEHITTVVVQTFRDLVWLCPQCTEAVCQALPGCEENIQDSEGKQALIWLLGVHGERIPNAPYVLEDFVENVKSETFPAVKMELLTALLRLFLSRPAECQDMLGRLLYYCIEEEKDMAVRDRGLFYYRLLLVGIDEVKRILCSPKSDPTLGLLEDPAERPVNSWASDFNTLVPVYGKAHWATISKCQGAERCDPELPKTSSFAASGPLIPEENKERVQELPDSGALMLVPNRQLTADYFEKTWLSLKVAHQQVLPWRGEFHPDTLQMALQVVNIQTIAMSRAGSRPWKAYLSAQDDTGCLFLTELLLEPGNSEMQISVKQNEARTETLNSFISVLETVIGTIEEIKS

SEQ ID NO: 3 - DNA, 3414 bp, Homo sapiens
atgagcgacatagtggagaagacgctgacggcgctgccgggactctttctgcagaaccagcccggtggtgggcccgcggccgccaaggcgtccttctcctcgaggctgggcagccttgtccgcggcatcacagccctcacctccaagcacgaagaagaaaaattaatccagcaggaactgagtagtctgaaagcgactgtttctgctcctactacaacactgaaaatgatgaaggaatgtatggtgagacttatatattgtgaaatgcttggatatgatgcttcctttggctatattcatgcaatcaagttagcccaacaaggaaacctcttagaaaaaagagtaggttatttggctgtttccttatttctacatgaaagtcatgaattattgcttctccttgtgaatacagttgtaaaggatctgcagagcactaacctagtagaagtgtgtatggcactgactgttgttagccagattttcccctgcgaaatgattccagctgttcttccattaatagaagataaacttcaacattctaaggagattgtacgaagaaaagctgttctggcattatacaaattccatctcattgctcctaatcaagtacaacatattcatattaagtttcggaaagcactttgtgacagagatgttggggtcatggctgcctccttgcatatatatcttagaatgattaaggagaattcatctggatataaagacttgactgggagttttgtaaccattttgaagcaagtagttggaggaaagctcccagtagaattcaattaccacagtgtgccagcaccatggttacaaattcagctcttgagaatactgggacttctaggaaaagatgatcaaaggacaagtgaattaatgtatgatgttcttgatgaatccttacgaagagctgagttaaatcacaatgtcacatatgctattttgtttgaatgtgtgcatacagtctattctatttatcctaaatcggaattacttgagaaggctgccaagtgcattggaaaatttgttctgtcacctaaaataaatctaaaatatttaggactgaaggctcttacctatgttatccaacaggatcctactctggctcttcaacaccagatgacaataattgaatgtttagatcatcctgatcccattattaaaagagagactctggaacttctttacagaattactaatgcacagaatataacagttattgtccagaaaatgcttgaatatttacatcagagcaaagaagagtatgtcatcgtcaatttggtcggcaaaatagcagagctggctgagaaatatgctcctgataatgcatggtttattcagacaatgaatgctgtgttttcagtaggaggagatgtaatgcatcctgatattcccaataactttctgagactactagcggaaggttttgatgatgaaacagaagatcagcaattaagactctatgcagttcagtcttatctcactttactggatatggaaaatgtgttctatccacagagatttcttcaagttatgagttgggtattaggggaatattcctacctcttagataaggaaacgccagaggaagttatagctaagctctacaagttacttatgaatgactctgtgtcttcagaaacaaaagcctggttaattgctgctgtgaccaaattgacatctcaggcgcactcttctaatacagttgagagattaatccatgaatttaccatatctttggatacttgtatgagacaacatgcatttgaattaaaacatttgcatgagaatgtggaacttatgaagagcttgcttccagttgacaggagttgtgaagacttggtggtagatgcttctttatcttttctggatggttttgtggctgaaggactcagtcagggtgcagcgccttacaaacctccccatcaacgccaggaggaaaagctttctcaggaaaaagttctcaattttgaaccatatggactctccttttcttcatctggcttcactggacgacagtctcctgctggcatttctcttggttcagatgtatctgggaatagtgctgagacaggactgaaagagacaaatagcttgaagctggaaggtataaagaaattgtgggggaaagaaggctatcttcccaagaaggaaagcaaaactggtgatgaaagtggagctctgcctgttcctcaagagagtataatggagaatgtagatcaagctataactaaaaaggatcaatctcaagttcttacccaatctaaagaggagaaagaaaagcagctgctggcatcatcattatttgttggtctaggatcagaaagtacaatcaacctgctgggaaaagcagatactgtctctcacaagttcagaaggaaatcaaaagtcaaagaagctaaaagtggcgaaacaaccagtactcataatatgacctgttcttcctttagttctttgtcaaatgtggcatatgaagatgattattattcgaatactttgcacgatacaggagacaaggaattaaagaaattttctctcacttcagaacttttggattctgagtcactcacagaactgcccttggttgagaaattctcatattgtagtctgtctacaccttcattgtttgctaataacaacatggaaatttttcaccctcctcaatctactgcagcctcagttgccaaggaaagctctttagcttcatcttttttggaagaaactactgaatacatacactcaaatgctatggaagtctgtaataatgaaactatatcagtgtcttcttataaaatttggaaagatgattgtttattgatggtctggtcagtcactaataagagtggtttggaattgaaaagtgctgacttagaaatttttcctgcagaaaatttcaaggtgactgagcaacctggatgctgtttgcctgtaatggaagcagaaagcaccaaaagctttcaatatagtgtgcagatagaaaaaccttttacagaaggaaatcttactggttttattagttatcatatgatggatactcattctgctcagctggaattttctgtaaacttatcactattagatttcattagaccattaaaaatctcaagtgacgactttgggaaactctggttatccttcgcaaatgatgtgaaacaaaatgtaaaaatgtcagaatctcaagctgcacttccttctgcactaaagactctgcaacagaaactaagactccatattattgagattataggcaatgaagggctattggcctgtcagctgctcccatccatcccctgcttactgcattgccgagttcatgcagatgtattagccctgtggttcagatcctcctgttctactcttcctgactatttactgtatcagtgtcaaaaggtgatggagggatcctag

SEQ ID NO: 4 - protein, 1137 aa, Homo sapiens
MSDIVEKTLTALPGLFLQNQPGGGPAAAKASFSSRLGSLVRGITALTSKHEEEKLIQQELSSLKATVSAPTTTLKMMKECMVRLIYCEMLGYDASFGYIHAIKLAQQGNLLEKRVGYLAVSLFLHESHELLLLLVNTVVKDLQSTNLVEVCMALTVVSQIFPCEMIPAVLPLIEDKLQHSKEIVRRKAVLALYKFHLIAPNQVQHIHIKFRKALCDRDVGVMAASLHIYLRMIKENSSGYKDLTGSFVTILKQVVGGKLPVEFNYHSVPAPWLQIQLLRILGLLGKDDQRTSELMYDVLDESLRRAELNHNVTYAILFECVHTVYSIYPKSELLEKAAKCIGKFVLSPKINLKYLGLKALTYVIQQDPTLALQHQMTIIECLDHPDPIIKRETLELLYRITNAQNITVIVQKMLEYLHQSKEEYVIVNLVGKIAELAEKYAPDNAWFIQTMNAVFSVGGDVMHPDIPNNFLRLLAEGFDDETEDQQLRLYAVQSYLTLLDMENVFYPQRFLQVMSWVLGEYSYLLDKETPEEVIAKLYKLLMNDSVSSETKAWLIAAVTKLTSQAHSSNTVERLIHEFTISLDTCMRQHAFELKHLHENVELMKSLLPVDRSCEDLVVDASLSFLDGFVAEGLSQGAAPYKPPHQRQEEKLSQEKVLNFEPYGLSFSSSGFTGRQSPAGISLGSDVSGNSAETGLKETNSLKLEGIKKLWGKEGYLPKKESKTGDESGALPVPQESIMENVDQAITKKDQSQVLTQSKEEKEKQLLASSLFVGLGSESTINLLGKADTVSHKFRRKSKVKEAKSGETTSTHNMTCSSFSSLSNVAYEDDYYSNTLHDTGDKELKKFSLTSELLDSESLTELPLVEKFSYCSLSTPSLFANNNMEIFHPPQSTAASVAKESSLASSFLEETTEYIHSNAMEVCNNETISVSSYKIWKDDCLLMVWSVTNKSGLELKSADLEIFPAENFKVTEQPGCCLPVMEAESTKSFQYSVQIEKPFTEGNLTGFISYHMMDTHSAQLEFSVNLSLLDFIRPLKISSDDFGKLWLSFANDVKQNVKMSESQAALPSALKTLQQKLRLHIIEIIGNEGLLACQLLPSIPCLLHCRVHADVLALWFRSSCSTLPDYLLYQCQKVMEGS

SEQ ID NO: 5 - DNA, 1362 bp, Homo sapiens
atgatttcccaattcttcattctgtcctccaagggggacccgctcatctacaaagacttccgcggggacagtggcggccgggatgtggccgagctcttctaccggaagctgacgggactgccaggagacgagtccccggttgtcatgcatcaccatggccgtcatttcattcacatcagacacagcggcctctatttggtggtcacaacttcagaaaacgtttctcccttcagcctcctagaactgctctccaggttggccacccttctgggcgattactgtggctccctgggcgaggggaccatctcccgcaatgtggctctggtatacgaactcctggatgaagtgctggactatggctatgtacagaccacatccacggagatgctgaggaatttcatccagacggaagctgtggtcagcaagcccttcagcctctttgacctcagcagcgttggcttgtttggggctgagacacaacagagcaaagtggcccccagcagtgcagccagccgccccgtcctgtccagtcgctctgaccagagccaaaagaatgaagtttttttggatgtggtcgagagattgtctgtactgatagcatctaatggatccctgctgaaggtggatgtgcagggagagattcggctcaagagcttccttcctagcggctctgagatgcgcattggcttgacggaagagttttgtgtggggaagtcagagctgagaggttatgggccaggaatccgggtcgatgaagtctcgtttcacagctctgtgaatctggacgaatttgagtctcatcgaatcctccgcttgcaaccacctcagggcgagctgactgtgatgcggtaccaactctccgatgacctcccctcaccgctccccttccggctcttcccctctgtgcagtgggaccgaggctcaggccggctccaggtttatctaaagttgcgatgtgacctgctctcaaagagccaagccctcaatgtcaggctgcacctccccctgcctcgaggggtggtcagcctgtctcaggagctgagcagcccagagcagaaggctgagctggcagagggagcccttcgctgggacctgcctcgggtgcaaggaggctctcaactctcaggccttttccagatggacgtcccagggcccccaggacctcccagccatgggctctccacctcggcctctcctctggggctgggccctgccagtctctccttcgagcttccccggcacacgtgctctggcctccaggtccgattcctcaggctggccttcaggccatgcggcaatgccaacccccacaagtgggtgcgacacctaagccacagcgacgcctatgtcattcggatctga

SEQ ID NO: 6 - protein, 453 aa, Homo sapiens
MISQFFILSSKGDPLIYKDFRGDSGGRDVAELFYRKLTGLPGDESPVVMHHHGRHFIHIRHSGLYLVVTTSENVSPFSLLELLSRLATLLGDYCGSLGEGTISRNVALVYELLDEVLDYGYVQTTSTEMLRNFIQTEAVVSKPFSLFDLSSVGLFGAETQQSKVAPSSAASRPVLSSRSDQSQKNEVFLDVVERLSVLIASNGSLLKVDVQGEIRLKSFLPSGSEMRIGLTEEFCVGKSELRGYGPGIRVDEVSFHSSVNLDEFESHRILRLQPPQGELTVMRYQLSDDLPSPLPFRLFPSVQWDRGSGRLQVYLKLRCDLLSKSQALNVRLHLPLPRGVVSLSQELSSPEQKAELAEGALRWDLPRVQGGSQLSGLFQMDVPGPPGPPSHGLSTSASPLGLGPASLSFELPRHTCSGLQVRFLRLAFRPCGNANPHKWVRHLSHSDAYVIRI

SEQ ID NO: 7 - DNA, 480 bp, Homo sapiens
atgataaaatttttcctcatggtgaataaacaagggcagactcgactttctaagtactatgaacatgtggatattaataagcgtacacttctggaaacagaagtcataaagagctgtctctctcgatccaatgaacaatgctctttcattgaatataaggattttaagctgatatatcggcagtatgcagctctcttcattgtggttggagttaatgacactgagaacgagatggctatttatgaattcattcataactttgtggaagttttagatgagtatttcagccgagtgagtgaattagatgtatcctttttcaatactgttttccacagtacttggcaaatgcactctggtccttatcaggaaccaattgatgaacttcccaaaatatgctcagccctggagccccagcagacttgcttttctccagacagttcatcattcaaaggagctgcctccaccacccccatctactga

SEQ ID NO: 8 - protein, 159 aa, Homo sapiens
MIKFFLMVNKQGQTRLSKYYEHVDINKRTLLETEVIKSCLSRSNEQCSFIEYKDFKLIYRQYAALFIVVGVNDTENEMAIYEFIHNFVEVLDEYFSRVSELDVSFFNTVFHSTWQMHSGPYQEPIDELPKICSALEPQQTCFSPDSSSFKGAASTTPIY

SEQ ID NO: 9 - DNA, 144 bp, artificial sequence
  CRISPR-sequence
ttggcgacgatgccataccttggctctgaggacgtggtgaaggaactgaagaaggctctgtgtaaccctcatattcaggctgataggctgcgctaccggaatgtcatccagcgagttattaggtatcaccaacctaccatagaa

SEQ ID NO: 10 - DNA, 19 bp, artificial sequence
  primer P1
tcgcccgaggacccaagaa

SEQ ID NO: 11 - DNA, 27 bp, artificial sequence
  primer
cctatcagcctgaatatgagggttaca

SEQ ID NO: 12 - DNA, 23 bp, artificial seqeunce
  primer
gctggatgacattccggtatatg

SEQ ID NO: 13 - DNA, 20 bp, artificial sequence
  primer
ctggtgaacgatgagaatgt

SEQ ID NO: 14 - DNA, 20 bp, artificial sequence
  primer
gacccagcaactctgttaaa

SEQ ID NO: 15 - DNA, 20 bp, artificial sequence
  primer
ctgtgctaggctcccacatc

SEQ ID NO: 16 - DNA, 20 bp, artificial sequence
  primer
tggcactggcctttaccatt

SEQ ID NO: 17 - DNA, 19 bp, artificial sequence
  primer
gtaacccgttgaaccccat

SEQ ID NO: 18 - DNA, 20 bp, artificial sequence
  primer
ccatccaatcggtagtagcg

SEQ ID NO: 19 - DNA, 6433 bp, artificial sequence
  hSYN1-hAPB1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaagtttgtacaaaaaagcaggctgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataacagagtcttgttgcttgtctagagtaagatgaataactattacactttcttagtctttcctattagagctgccagtaagtccagataatatcagatacaaaggagagtggaaaatctagaaatcaggattcttaagtttttggccaaagaacattgattcattcctttcattcctattggcaattgaccccatttttcagtgatgatgttgtagaggttaatgttggtaaggagatgcgttgaggatgggatttggattatttcctaatcaattttaggaattgtgttagtaattaaagatggctgatgtgtggaaactgataggggtcatcatttgtgggggtcatcttttcttccctggattattatagtaaattcctaactgttctcatatttccaatcatgcttttaggccagattccaaagtccttaatgtcatctaaagtacctctttggtgtcaagtcttgccactcccaaaatgacttcttttctggcagaagcaaaccatttgcattgcagttccctttatacacctgcttctttcacctctgtgttaacttaaacctgcttttatttattgcctggaaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 20 - DNA, 6174 bp, artificial sequence
  CBh-hAP4B1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 21 - DNA, 5255 bp, artificial sequence
  SYN1hAP4S1
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcaactttgtatagaaaagttgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaagtttgtacaaaaaagcaggctgccaccatgataaaatttttcctcatggtgaataaacaagggcagactcgactttctaagtactatgaacatgtggatattaataagcgtacacttctggaaacagaagtcataaagagctgtctctctcgatccaatgaacaatgctctttcattgaatataaggattttaagctgatatatcggcagtatgcagctctcttcattgtggttggagttaatgacactgagaacgagatggctatttatgaattcattcataactttgtggaagttttagatgagtatttcagccgagtgagtgaattagatataatgtttaatttggataaagtacacatcattttggatgagatggtgttaaatggctgcattgtggaaactaacagggcaagaattcttgcccctctactaattcttgataagatgtcagaaagctgaaaggaagtctcttcgagacaatatggatttatcagaaatgcgagtaccgtggaatacatctcaacatgttaacccagaagaatctggaagaccacaattacaaaatggggtatccttccaaagacattataaataggcattttccacagttcctaaaaagaaaacacaactgtactttaaaatatgtacaaagaaaaaaatttctttaaactgagagagaagttttattttctaattgtaaacatatctgtcgcactttaaattctgttgagcacctaaggaacccttcttggtctatgcttcttttgcaaattgaattcaggaatagcaggatggtagtgggaagaaagtatggcagttttccgtcagccaataaagttttaaaatttaaacacaaggcattttgagtaacgctgttttctgaaagctatttttttttttttgagacggagtctagtctagctgtgtcacctggcacattctcggctcactgcaacctccacttccggggttcaagcgattctcctgcctcagcctccgagctgggactacaggcatgcaccaccacacctggctaatttttgtatttttagtagagacagggtttcaccatgttggccaggctgtctcaaactcctgacctcaggtgatctgcctgcctcagcctcccagaacgctgggattacagatgagccacagtgcctgacttctgaaggctgtcttattcctttaaaaggcttagtaaaattaactttttaagaggtgtttttttgtttgtttttttgttttgacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgactgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 22 - DNA, 3909 bp, artificial sequence
  MeP229hAP4S1
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggcaactttgtatagaaaagttgcaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaacaagtttgtacaaaaaagcaggctgccaccatgataaaatttttcctcatggtgaataaacaagggcagactcgactttctaagtactatgaacatgtggatattaataagcgtacacttctggaaacagaagtcataaagagctgtctctctcgatccaatgaacaatgctctttcattgaatataaggattttaagctgatatatcggcagtatgcagctctcttcattgtggttggagttaatgacactgagaacgagatggctatttatgaattcattcataactttgtggaagttttagatgagtatttcagccgagtgagtgaattagatataatgtttaatttggataaagtacacatcattttggatgagatggtgttaaatggctgcattgtggaaactaacagggcaagaattcttgcccctctactaattcttgataagatgtcagaaagctgaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctg

SEQ ID NO: 23 - DNA, 6795 bp, artificial sequence
  MeP229hAP4E1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaacaagtttgtacaaaaaagcaggctgccaccatgagcgacatagtggagaagacgctgacggcgctgccgggactctttctgcagaaccagcccggtggtgggcccgcggccgccaaggcgtccttctcctcgaggctgggcagccttgtccgcggcatcacagccctcacctccaagcacgaagaagaaaaattaatccagcaggaactgagtagtctgaaagcgactgtttctgctcctactacaacactgaaaatgatgaaggaatgtatggtgagacttatatattgtgaaatgcttggatatgatgcttcctttggctatattcatgcaatcaagttagcccaacaaggaaacctcttagaaaaaagagtaggttatttggctgtttccttatttctacatgaaagtcatgaattattgcttctccttgtgaatacagttgtaaaggatctgcagagcactaacctagtagaagtgtgtatggcactgactgttgttagccagattttcccctgcgaaatgattccagctgttcttccattaatagaagataaacttcaacattctaaggagattgtacgaagaaaagctgttctggcattatacaaattccatctcattgctcctaatcaagtacaacatattcatattaagtttcggaaagcactttgtgacagagatgttggggtcatggctgcctccttgcatatatatcttagaatgattaaggagaattcatctggatataaagacttgactgggagttttgtaaccattttgaagcaagtagttggaggaaagctcccagtagaattcaattaccacagtgtgccagcaccatggttacaaattcagctcttgagaatactgggacttctaggaaaagatgatcaaaggacaagtgaattaatgtatgatgttcttgatgaatccttacgaagagctgagttaaatcacaatgtcacatatgctattttgtttgaatgtgtgcatacagtctattctatttatcctaaatcggaattacttgagaaggctgccaagtgcattggaaaatttgttctgtcacctaaaataaatctaaaatatttaggactgaaggctcttacctatgttatccaacaggatcctactctggctcttcaacaccagatgacaataattgaatgtttagatcatcctgatcccattattaaaagagagactctggaacttctttacagaattactaatgcacagaatataacagttattgtccagaaaatgcttgaatatttacatcagagcaaagaagagtatgtcatcgtcaatttggtcggcaaaatagcagagctggctgagaaatatgctcctgataatgcatggtttattcagacaatgaatgctgtgttttcagtaggaggagatgtaatgcatcctgatattcccaataactttctgagactactagcggaaggttttgatgatgaaacagaagatcagcaattaagactctatgcagttcagtcttatctcactttactggatatggaaaatgtgttctatccacagagatttcttcaagttatgagttgggtattaggggaatattcctacctcttagataaggaaacgccagaggaagttatagctaagctctacaagttacttatgaatgactctgtgtcttcagaaacaaaagcctggttaattgctgctgtgaccaaattgacatctcaggcgcactcttctaatacagttgagagattaatccatgaatttaccatatctttggatacttgtatgagacaacatgcatttgaattaaaacatttgcatgagaatgtggaacttatgaagagcttgcttccagttgacaggagttgtgaagacttggtggtagatgcttctttatcttttctggatggttttgtggctgaaggactcagtcagggtgcagcgccttacaaacctccccatcaacgccaggaggaaaagctttctcaggaaaaagttctcaattttgaaccatatggactctccttttcttcatctggcttcactggacgacagtctcctgctggcatttctcttggttcagatgtatctgggaatagtgctgagacaggactgaaagagacaaatagcttgaagctggaaggtataaagaaattgtgggggaaagaaggctatcttcccaagaaggaaagcaaaactggtgatgaaagtggagctctgcctgttcctcaagagagtataatggagaatgtagatcaagctataactaaaaaggatcaatctcaagttcttacccaatctaaagaggagaaagaaaagcagctgctggcatcatcattatttgttggtctaggatcagaaagtacaatcaacctgctgggaaaagcagatactgtctctcacaagttcagaaggaaatcaaaagtcaaagaagctaaaagtggcgaaacaaccagtactcataatatgacctgttcttcctttagttctttgtcaaatgtggcatatgaagatgattattattcgaatactttgcacgatacaggagacaaggaattaaagaaattttctctcacttcagaacttttggattctgagtcactcacagaactgcccttggttgagaaattctcatattgtagtctgtctacaccttcattgtttgctaataacaacatggaaatttttcaccctcctcaatctactgcagcctcagttgccaaggaaagctctttagcttcatcttttttggaagaaactactgaatacatacactcaaatgctatggaagtctgtaataatgaaactatatcagtgtcttcttataaaatttggaaagatgattgtttattgatggtctggtcagtcactaataagagtggtttggaattgaaaagtgctgacttagaaatttttcctgcagaaaatttcaaggtgactgagcaacctggatgctgtttgcctgtaatggaagcagaaagcaccaaaagctttcaatatagtgtgcagatagaaaaaccttttacagaaggaaatcttactggttttattagttatcatatgatggatactcattctgctcagctggaattttctgtaaacttatcactattagatttcattagaccattaaaaatctcaagtgacgactttgggaaactctggttatccttcgcaaatgatgtgaaacaaaatgtaaaaatgtcagaatctcaagctgcacttccttctgcactaaagactctgcaacagaaactaagactccatattattgagattataggcaatgaagggctattggcctgtcagctgctcccatccatcccctgcttactgcattgccgagttcatgcagatgtattagccctgtggttcagatcctcctgttctactcttcctgactatttactgtatcagtgtcaaaaggtgatggagggatcctagacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 24 - DNA, 7039 bp, artificial sequence
  SYN1hAP4E1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaagtttgtacaaaaaagcaggctgccaccatgagcgacatagtggagaagacgctgacggcgctgccgggactctttctgcagaaccagcccggtggtgggcccgcggccgccaaggcgtccttctcctcgaggctgggcagccttgtccgcggcatcacagccctcacctccaagcacgaagaagaaaaattaatccagcaggaactgagtagtctgaaagcgactgtttctgctcctactacaacactgaaaatgatgaaggaatgtatggtgagacttatatattgtgaaatgcttggatatgatgcttcctttggctatattcatgcaatcaagttagcccaacaaggaaacctcttagaaaaaagagtaggttatttggctgtttccttatttctacatgaaagtcatgaattattgcttctccttgtgaatacagttgtaaaggatctgcagagcactaacctagtagaagtgtgtatggcactgactgttgttagccagattttcccctgcgaaatgattccagctgttcttccattaatagaagataaacttcaacattctaaggagattgtacgaagaaaagctgttctggcattatacaaattccatctcattgctcctaatcaagtacaacatattcatattaagtttcggaaagcactttgtgacagagatgttggggtcatggctgcctccttgcatatatatcttagaatgattaaggagaattcatctggatataaagacttgactgggagttttgtaaccattttgaagcaagtagttggaggaaagctcccagtagaattcaattaccacagtgtgccagcaccatggttacaaattcagctcttgagaatactgggacttctaggaaaagatgatcaaaggacaagtgaattaatgtatgatgttcttgatgaatccttacgaagagctgagttaaatcacaatgtcacatatgctattttgtttgaatgtgtgcatacagtctattctatttatcctaaatcggaattacttgagaaggctgccaagtgcattggaaaatttgttctgtcacctaaaataaatctaaaatatttaggactgaaggctcttacctatgttatccaacaggatcctactctggctcttcaacaccagatgacaataattgaatgtttagatcatcctgatcccattattaaaagagagactctggaacttctttacagaattactaatgcacagaatataacagttattgtccagaaaatgcttgaatatttacatcagagcaaagaagagtatgtcatcgtcaatttggtcggcaaaatagcagagctggctgagaaatatgctcctgataatgcatggtttattcagacaatgaatgctgtgttttcagtaggaggagatgtaatgcatcctgatattcccaataactttctgagactactagcggaaggttttgatgatgaaacagaagatcagcaattaagactctatgcagttcagtcttatctcactttactggatatggaaaatgtgttctatccacagagatttcttcaagttatgagttgggtattaggggaatattcctacctcttagataaggaaacgccagaggaagttatagctaagctctacaagttacttatgaatgactctgtgtcttcagaaacaaaagcctggttaattgctgctgtgaccaaattgacatctcaggcgcactcttctaatacagttgagagattaatccatgaatttaccatatctttggatacttgtatgagacaacatgcatttgaattaaaacatttgcatgagaatgtggaacttatgaagagcttgcttccagttgacaggagttgtgaagacttggtggtagatgcttctttatcttttctggatggttttgtggctgaaggactcagtcagggtgcagcgccttacaaacctccccatcaacgccaggaggaaaagctttctcaggaaaaagttctcaattttgaaccatatggactctccttttcttcatctggcttcactggacgacagtctcctgctggcatttctcttggttcagatgtatctgggaatagtgctgagacaggactgaaagagacaaatagcttgaagctggaaggtataaagaaattgtgggggaaagaaggctatcttcccaagaaggaaagcaaaactggtgatgaaagtggagctctgcctgttcctcaagagagtataatggagaatgtagatcaagctataactaaaaaggatcaatctcaagttcttacccaatctaaagaggagaaagaaaagcagctgctggcatcatcattatttgttggtctaggatcagaaagtacaatcaacctgctgggaaaagcagatactgtctctcacaagttcagaaggaaatcaaaagtcaaagaagctaaaagtggcgaaacaaccagtactcataatatgacctgttcttcctttagttctttgtcaaatgtggcatatgaagatgattattattcgaatactttgcacgatacaggagacaaggaattaaagaaattttctctcacttcagaacttttggattctgagtcactcacagaactgcccttggttgagaaattctcatattgtagtctgtctacaccttcattgtttgctaataacaacatggaaatttttcaccctcctcaatctactgcagcctcagttgccaaggaaagctctttagcttcatcttttttggaagaaactactgaatacatacactcaaatgctatggaagtctgtaataatgaaactatatcagtgtcttcttataaaatttggaaagatgattgtttattgatggtctggtcagtcactaataagagtggtttggaattgaaaagtgctgacttagaaatttttcctgcagaaaatttcaaggtgactgagcaacctggatgctgtttgcctgtaatggaagcagaaagcaccaaaagctttcaatatagtgtgcagatagaaaaaccttttacagaaggaaatcttactggttttattagttatcatatgatggatactcattctgctcagctggaattttctgtaaacttatcactattagatttcattagaccattaaaaatctcaagtgacgactttgggaaactctggttatccttcgcaaatgatgtgaaacaaaatgtaaaaatgtcagaatctcaagctgcacttccttctgcactaaagactctgcaacagaaactaagactccatattattgagattataggcaatgaagggctattggcctgtcagctgctcccatccatcccctgcttactgcattgccgagttcatgcagatgtattagccctgtggttcagatcctcctgttctactcttcctgactatttactgtatcagtgtcaaaaggtgatggagggatcctagacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 25 - DNA, 588 bp, artificial sequence
  stuffer sequence
cagagtcttgttgcttgtctagagtaagatgaataactattacactttcttagtctttcctattagagctgccagtaagtccagataatatcagatacaaaggagagtggaaaatctagaaatcaggattcttaagtttttggccaaagaacattgattcattcctttcattcctattggcaattgaccccatttttcagtgatgatgttgtagaggttaatgttggtaaggagatgcgttgaggatgggatttggattatttcctaatcaattttaggaattgtgttagtaattaaagatggctgatgtgtggaaactgataggggtcatcatttgtgggggtcatcttttcttccctggattattatagtaaattcctaactgttctcatatttccaatcatgcttttaggccagattccaaagtccttaatgtcatctaaagtacctctttggtgtcaagtcttgccactcccaaaatgacttcttttctggcagaagcaaaccatttgcattgcagttccctttatacacctgcttctttcacctctgtgttaacttaaacctgcttttatttattgcctggaa

SEQ ID NO: 26 - DNA, 790 bp, artificial sequence
  stuffer sequence
aaggaagtctcttcgagacaatatggatttatcagaaatgcgagtaccgtggaatacatctcaacatgttaacccagaagaatctggaagaccacaattacaaaatggggtatccttccaaagacattataaataggcattttccacagttcctaaaaagaaaacacaactgtactttaaaatatgtacaaagaaaaaaatttctttaaactgagagagaagttttattttctaattgtaaacatatctgtcgcactttaaattctgttgagcacctaaggaacccttcttggtctatgcttcttttgcaaattgaattcaggaatagcaggatggtagtgggaagaaagtatggcagttttccgtcagccaataaagttttaaaatttaaacacaaggcattttgagtaacgctgttttctgaaagctatttttttttttttgagacggagtctagtctagctgtgtcacctggcacattctcggctcactgcaacctccacttccggggttcaagcgattctcctgcctcagcctccgagctgggactacaggcatgcaccaccacacctggctaatttttgtatttttagtagagacagggtttcaccatgttggccaggctgtctcaaactcctgacctcaggtgatctgcctgcctcagcctcccagaacgctgggattacagatgagccacagtgcctgacttctgaaggctgtcttattcctttaaaaggcttagtaaaattaactttttaagaggtgtttttttgtttgtttttttgttttg

SEQ ID NO: 27 - DNA, 600 bp, artificial sequence
  Ap4B1 promoter
ggcttccacaacggcgtcccgataaaagtcatgctggggcaagaacagggccagagaccctaagggaaagagcccagcagagcaataagttaccagagtgggcagaaaaaggaagtcaccctgcccgctggagcccaaatcgcggagggcgctggacactcccaaccgcgcgggagcggaggccgagccgggcactcaaagcgcgcgctgcagggaggagacaggccaaagtagccgatttccgattgcccgccgcagctccaccagagggagccgcgcagctgcgcctggagagtgagaggttgggggaagggcatcctaaggtcggagtcgggactacgcttagcgctcgctcgcggggagaccggcagcggtcttctgtttggtctcttgacaagggggcgggacttccgcagaaagcaagattgggcctatccgcgatgagaacggcgtggagggggaattaaggctggaactggagagggaggggtctgcggtcagtcggacgcggcgctggaaatccggcggaggacctagaagtagcgggagcgatctgtgggcggggcgaacggcctgccggccgacggtagggcctgaaga

SEQ ID NO: 28 - DNA, 21 bp, artificial sequence
  AP4 promoter forward primer sequence
cgtggcttccacaacggcgtc

SEQ ID NO: 29 - DNA, 30 bp, artificial sequence
  AP4 Promoter reverse primer sequence
ggctgccatcccggacttctcttaaggcta

SEQ ID NO: 30 - DNA, 60 bp, artificial sequence
  minimal promoter sequence
cgtggagggggaattaaggctggaactggagagggaggggtctgcggtcagtcggacgcg